SEQUENCE LISTING (48 sequences)

SEQ ID NO: 1 - DNA, 339 bp, synthetic construct
  misc_feature 1..339: note = Synthetic polypeptide
  source 1..339: mol_type = other DNA
atgaaaaaattattattcgcaattcctttatgtctcagctacgaaaccgaaatcttgaccgtcgaatatggtctgctgccaatcggcaagattgttgaaaaacgtattgaatgtacggtctactcagtggataacaacggcaatatctacacccagccggtggcccagtggcatgaccgtggtgaacaggaagtgttcgaatattgtctggaagacggatctttaatccgtgccacaaaggatcacaaatttatgactgtagatggtcagatgctcccaatcgacgaaatttttgaacgcgaattagacctgatgcgcgtggataatctcccgaattaa

SEQ ID NO: 2 - DNA, 1362 bp, synthetic construct
  misc_feature 1..1362: note = Synthetic polypeptide
  source 1..1362: mol_type = other DNA
atgatcaaaattgccacgcgtaaatatttaggcaaacagaatgtttatgatatcggtgtcgagcgcgatcataatttcgcgctgaaaaacggctttatcgccagcaattgttttaatgttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccttacgagttcagtatcgactgcgataagatcaacctgttccgcggtgtctttgcgtttcttttatatgttgccacctttatgtatgtattttctacgtttgctaacatactgcgtaataaggagtcttaa

SEQ ID NO: 3 - DNA, 534 bp, synthetic construct
  misc_feature 1..534: note = Synthetic polypeptide
  source 1..534: mol_type = other DNA
aacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaag

SEQ ID NO: 4 - DNA, 534 bp, synthetic construct
  misc_feature 1..534: note = Synthetic polypeptide
  source 1..534: mol_type = other DNA
aacaccattaatatcgctaagaacggcttctctgatatcgaactggctgctatcccgttcaacactctggctgaccattacggtgagcgtttagctcgcgaacagttggcccttgagcatgagtcttacgagatggatgaagcacgcttccgcaagatgtttgagcgtcaacttaaagctggtgaggttgcggataacactgccgccaatcctctcatcactaccctactccttaagatgattgcacgcatcaaggactggtttgaggaagtgaaagctaagcgcggcaagcgcccgacagccttccatttcctgaaagaaatcaagccggaagccgtagcgtacatcaccattaaggccactctggcttgcctaaccagtgctaacaaaacaaccgttcaggctgtagcaagcgcaatcggtcgggccattgaggacgaggctcgcttcggtcgtatccgttaccttgaagctaagcacttcaagaaaaacgttgaggaacaactcaacaagcgcgtagggcacgtccacaag

SEQ ID NO: 5 - DNA, 105 bp, synthetic construct
  misc_feature 1..105: note = Synthetic polypeptide
  source 1..105: mol_type = other DNA
ttgcaaagaatgaaacaacttgaaccgaaggttgaagaattgcttccgaaaaattatcacttggaaaatgaggttgccagattaaagaaattagttggcgaacgc

SEQ ID NO: 6 - DNA, 684 bp, synthetic construct
  misc_feature 1..684: note = Synthetic polypeptide
  source 1..684: mol_type = other DNA
tcttctgaaaccggtccggttgcggttgacccgaccctgcgtcgtcgtatcgaaccgcacgaattcgaagttttcttcgacccgcgtgaactgcgtaaagaaacctgcctgctgtacgaaatcaactggggtggtcgtcactctatctggcgtcacacctctcagaacaccaacaaacacgttgaagttaacttcatcgaaaaattcaccaccgaacgttacttctgcccgaacacccgttgctctatcacctggttcctgtcttggtctccgtgcggtgaatgctctcgtgcgatcaccgaattcctgtctcgttacccgcacgttaccctgttcatctacatcgcgcgtctgtaccaccacgcggacccgcgtaaccgtcagggtctgcgtgacctgatctcttctggtgttaccatccagatcatgaccgaacaggaatctggttactgctggcgtaacttcgttaactactctccgtctaacgaagcgcactggccgcgttacccgcacctgtgggttcgtctgtacgttctggaactgtactgcatcatcctgggtctgccgccgtgcctgaacatcctgcgtcgtaaacagccgcagctgaccttcttcaccatcgcgctgcagtcttgccactaccagcgtctgccgccgcacatcctgtgggcgaccggtctgaaa

SEQ ID NO: 7 - DNA, 684 bp, synthetic construct
  misc_feature 1..684: note = Synthetic polypeptide
  source 1..684: mol_type = other DNA
tcttctgaaaccggtccggttgcggttgacccgaccctgcgtcgtcgtatcgaaccgcacgaattcgaagttttcttcgacccgcgtgaactgcgtaaagaaacctgcctgctgtacgaaatcaactggggtggtcgtcactctatctggcgtcacacctctcagaacaccaacaaacacgttgaagttaacttcatcgaaaaattcaccaccgaacgttacttctgcccgaacacccgttgctctatcacctggttcctgtcttggtctccgtgcggtgaatgctctcgtgcgatcaccgaattcctgtctcgttacccgcacgttaccctgtgcatctacatcgcgcgtctgtaccaccacgaggacccgcgtaaccgtcagggtctgcgtgacctgatctcttctggtgttaccatccagatcatgaccgaacaggaatctggttactgctggcgtaacttcgttaactactctccgtctaacgaagcgcactggccgcgttacccgcacctgtgggttcgtctgtacgttctggaactgtactgcatcatcctgggtctgccgccgtgcctgaacatcctgcgtcgtaaacagccgcagctgacctccttcaccatcgcgctgcagtcttgccactaccagcgtctgccgccgcacatcctgtgggcgaccggtctgaaa

SEQ ID NO: 8 - DNA, 684 bp, synthetic construct
  misc_feature 1..684: note = Synthetic polypeptide
  source 1..684: mol_type = other DNA
tcttctgaaaccggtccggttgcggttgacccgaccctgcgtcgtcgtatcgaaccgcacgaattcgaagttttcttcgacccgcgtgaactgcgtaaagaaacctgcctgctgtacgaaatcaactgggatggtcgtcactctatctggcgtcacacctctcagaacaccaacaaacacgttgaagttaacttcatcgaaaaattcaccaccgaacgtcacttctgccctaacacccgttgctctatcacctggttcctgtcttggtctccgtgcggtgaatgctctcgtgcgatcaccgaattcctgtctcgttacccgcacgttaccctgtgcatctacatcgcgcgtctgtaccaccacgaggacccgcgtaaccgtcagggtctgcgtgacctgatctcttctggtgttaccatccagatcatgaccgaacaggaatctggttactgctggcgtaacttcgttaactactctccgtctaacgaagcgaactggccgcgttacccgcacctgtgggttcgtctgtacgttctggaactgtactgcatcatcctgggtctgccgccgtgcctgaacatcctgcgtcgtaaacagccgcagctgacctccttcaccatcgcgctgcagtcttgccactaccagcgtctgccgccgcacatcctgtgggcgcccggtctgaaa

SEQ ID NO: 9 - DNA, 684 bp, synthetic construct
  misc_feature 1..684: note = Synthetic polypeptide
  source 1..684: mol_type = other DNA
tcttctgaaaccggtccggttgcggttgacccgaccctgcgtcgtcgtatcgaaccgcacgaattcgaagttttcttcgacccgcgtgaactgcgtaaagaaacctgtctgctgtacgaaatcaactggggtggtcgtcactctatctggcgtcacacctctcagaacaccaacaaacacgttgcagttaacttcatcgaaaaattcaccaccgaacgttacttctgcccgaacacccgttgctctatcacctggttcctgtcttggtctccgtgcggtgaatgctctcgtgcgatcaccgaattcctgtctcgttacccgcacgttaccctgtgcatctacatcgcgcgtctgtaccaccacgaggacccgcgtaaccgtcagggtctgcgtgacctgatctcttctggtgttaccatccagatcatgaccgaacaggaatctggttactgctggcgtaacttcgttaactactctccgtctaacgaaacgcactggccgcgttacccgcacctgtgggttcgtctgtacgttctggaactgtactgcatcatcctgggtctgccgccgtgcctgaacatcctgcgtcgtaaacagccgcagctgacctccttcaccatcgcgctgcagtcttgccactaccagcgtctgccgccgcacatcctgtgggcgaccggtctgaaa

SEQ ID NO: 10 - DNA, 684 bp, synthetic construct
  misc_feature 1..684: note = Synthetic polypeptide
  source 1..684: mol_type = other DNA
tcttctgaaaccggtccggttgcggttgacccgaccctgcgtcgtcgtatcgaaccgcacgaattcgaagttttcttcgacccgcgtgaactgcgtaaagaaacctgtctgctgtacgaaatcaactggggtggtcgtcactctatctggcgtcacacctctcagaacaccaacaaacacgttgaagttaacttcatcgaaaaattcaccaccgaacgttacttctgcccgaacacccgttgctctatcacctggttcctgtcttggtctccgtgcggtgaatgctctcgtgcgatcaccgaattcctgtctcgttacccgcacgttaccctgtgcatctacatcgcgcgtctgtaccaccacgaggacccgcgtaaccgtcagggtctgcgtgacctgatctcttctggtgttaccatccagatcatgaccgaacaggaatctggttactgctggcgtaacttcgttaactactctccgtctaacgaaacgcactggccgcgttacccgcacctgtgggttcgtctgtacgttctggaactgtactgcatcatcctgggtctgccgccgtgcctgaacatcctgcgtcgtaaacagccgcagctgacctccttcaccatcgcgctgcagtcttgccactaccagcgtctgccgccgcacatcctgtgggcgaccggtctgaaa

SEQ ID NO: 11 - DNA, 684 bp, synthetic construct
  misc_feature 1..684: note = Synthetic polypeptide
  source 1..684: mol_type = other DNA
agctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaa

SEQ ID NO: 12 - DNA, 684 bp, synthetic construct
  misc_feature 1..684: note = Synthetic polypeptide
  source 1..684: mol_type = other DNA
agctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtgtatttacatcgcaaggctgtaccaccacgaagacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacatcctttaccatcgctcttcagtcttgtcattaccagcgactgcccccacacattctctgggccaccgggttgaaa

SEQ ID NO: 13 - DNA, 684 bp, synthetic construct
  misc_feature 1..684: note = Synthetic polypeptide
  source 1..684: mol_type = other DNA
agctcagagactggcccagtggctgtggaccccacattgagacggcggatcgagccccatgagtttgaggtattcttcgatccgagagagctccgcaaggagacctgcctgctttacgaaattaattgggatggccggcactccatttggcgacatacatcacagaacactaacaagcacgtcgaagtcaacttcatcgagaagttcacgacagaaagacacttctgtccgaacacaaggtgcagcattacctggtttctcagctggagcccatgcggcgaatgtagtagggccatcactgaattcctgtcaaggtatccccacgtcactctgtgtatttacatcgcaaggctgtaccaccacgaagacccccgcaatcgacaaggcctgcgggatttgatctcttcaggtgtgactatccaaattatgactgagcaggagtcaggatactgctggagaaactttgtgaattatagcccgagtaatgaagccaactggcctaggtatccccatctgtgggtacgactgtacgttcttgaactgtactgcatcatactgggcctgcctccttgtctcaacattctgagaaggaagcagccacagctgacatcctttaccatcgctcttcagtcttgtcattaccagcgactgcccccacacattctctgggccaccgggttgaaa

SEQ ID NO: 14 - DNA, 684 bp, synthetic construct
  misc_feature 1..684: note = Synthetic polypeptide
  source 1..684: mol_type = other DNA
agctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtgtatttacatcgcaaggctgtaccaccacgaagacccccgcaatcgacaaggcctgcgggatttgatctcttcaggtgtgactatccaaattatgactgagcaggagtcaggatactgctggagaaactttgtgaattatagcccgagtaatgaaacccactggcctaggtatccccatctgtgggtacgactgtacgttcttgaactgtactgcatcatactgggcctgcctccttgtctcaacattctgagaaggaagcagccacagctgacatcctttaccatcgctcttcagtcttgtcattaccagcgactgcccccacacattctctgggccaccgggttgaaa

SEQ ID NO: 15 - protein, 229 aa, synthetic construct [sequence omitted]

SEQ ID NO: 16 - protein, 229 aa, synthetic construct
  REGION 1..229: note = Synthetic polypeptide
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLCIYIARLYHHEDPRNRQGLRDLISSGVTIQIMTEQESGYCWRNFVNYSPSNEAHWPRYPHLWVRLYVLELYCIILGLPPCLNILRRKQPQLTSFTIALQSCHYQRLPPHILWATGLK

SEQ ID NO: 17 - protein, 229 aa, synthetic construct
  REGION 1..229: note = Synthetic polypeptide
MSSETGPVAVDPTLRRRIEPHEFEVFFDPRELRKETCLLYEINWDGRHSIWRHTSQNTNKHVEVNFIEKFTTERHFCPNTRCSITWFLSWSPCGECSRAITEFLSRYPHVTLCIYIARLYHHEDPRNRQGLRDLISSGVTIQIMTEQESGYCWRNFVNYSPSNEANWPRYPHLWVRLYVLELYCIILGLPPCLNILRRKQPQLTSFTIALQSCHYQRLPPHILWAPGLK

SEQ ID NO: 18 - protein, 229 aa, synthetic construct
  REGION 1..229: note = Synthetic polypeptide
MSSETGPVAVDPTLRRRIEPHEFEVFFDPRELRKETCLLYEINWGGRHSIWRHTSQNTNKHVAVNFIEKFTTERYFCPNTRCSITWFLSWSPCGECSRAITEFLSRYPHVTLCIYIARLYHHEDPRNRQGLRDLISSGVTIQIMTEQESGYCWRNFVNYSPSNETHWPRYPHLWVRLYVLELYCIILGLPPCLNILRRKQPQLTSFTIALQSCHYQRLPPHILWATGLK

SEQ ID NO: 19 - protein, 229 aa, synthetic construct
  REGION 1..229: note = Synthetic polypeptide
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLCIYIARLYHHEDPRNRQGLRDLISSGVTIQIMTEQESGYCWRNFVNYSPSNETHWPRYPHLWVRLYVLELYCIILGLPPCLNILRRKQPQLTSFTIALQSCHYQRLPPHILWATGLK

SEQ ID NO: 20 - protein, 1367 aa, synthetic construct
  REGION 1..1367: note = Synthetic polypeptide
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 21 - protein, 1367 aa, synthetic construct
  REGION 1..1367: note = Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 22 - protein, 84 aa, synthetic construct
  REGION 1..84: note = Synthetic polypeptide
MTNLSDIIEKETGKQLVIQESILMLPEEVEEVIGNKPESDILVHTAYDESTDENVMLLTSDAPEYKPWALVIQDSNGENKIKML

SEQ ID NO: 23 - DNA, 4104 bp, Streptococcus pyogenes
  source 1..4104: mol_type = genomic DNA
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttggcagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggcagattctactgataaagcggatttgcgcttaatctatttggccttagcgcatatgattaagtttcgtggtcattttttgattgagggagatttaaatcctgataatagtgatgtggacaaactatttatccagttggtacaaatctacaatcaattatttgaagaaaaccctattaacgcaagtagagtagatgctaaagcgattctttctgcacgattgagtaaatcaagacgattagaaaatctcattgctcagctccccggtgagaagagaaatggcttgtttgggaatctcattgctttgtcattgggattgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatagtgaaataactaaggctcccctatcagcttcaatgattaagcgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggcgcctaccatgatttgctaaaaattattaaagataaagattttttggataatgaagaaaatgaagatatcttagaggatattgttttaacattgaccttatttgaagatagggggatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagatattcaaaaagcacaggtgtctggacaaggccatagtttacatgaacagattgctaacttagctggcagtcctgctattaaaaaaggtattttacagactgtaaaaattgttgatgaactggtcaaagtaatggggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctacaaaatggaagagacatgtatgtggaccaagaattagatattaatcgtttaagtgattatgatgtcgatcacattgttccacaaagtttcattaaagacgattcaatagacaataaggtactaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 24 - protein, 1367 aa, Streptococcus pyogenes
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 25 - DNA, 4212 bp, synthetic construct
  misc_feature 1..4212: note = Synthetic polypeptide
  source 1..4212: mol_type = other DNA
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcagga

SEQ ID NO: 26 - protein, 1368 aa, synthetic construct
  REGION 1..1368: note = Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 27 - DNA, 4107 bp, Streptococcus pyogenes
  source 1..4107: mol_type = genomic DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 28 - protein, 1368 aa, Streptococcus pyogenes [sequence omitted]

SEQ ID NO: 29 - protein, 1368 aa, synthetic construct
  REGION 1..1368: note = Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 30 - protein, 236 aa, synthetic construct
  REGION 1..236: note = Synthetic polypeptide
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 31 - protein, 120 aa, synthetic construct
  REGION 1..120: note = Synthetic polypeptide
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 32 - protein, 150 aa, synthetic construct
  REGION 1..150: note = Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 33 - protein, 150 aa, synthetic construct
  REGION 1..150: note = Synthetic polypeptide
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 34 - protein, 16 aa, synthetic construct
  REGION 1..16: note = Synthetic polypeptide
SGSETPGTSESATPES

SEQ ID NO: 35 - protein, 7 aa, synthetic construct
  REGION 1..7: note = Synthetic polypeptide
PKKKRKV

SEQ ID NO: 36 - protein, 30 aa, synthetic construct
  REGION 1..30: note = Synthetic polypeptide
MDSLLMNRRKFLYQFKNVRWAKGRRETYLC

SEQ ID NO: 37 - RNA, 81 nt, synthetic construct
  misc_feature 1..81: note = Synthetic polypeptide
  source 1..81: mol_type = other RNA
gttttagagctagaaatagcaagttaaaataaggctagtccgttatcaacttgaaaaagtggcaccgagtcggtgcttttt

SEQ ID NO: 38 - protein, 9 aa, synthetic construct
  REGION 1..9: note = Synthetic polypeptide
MKKLLPAIP

SEQ ID NO: 39 - protein, 414 aa, synthetic construct
  REGION 1..414: note = Synthetic polypeptide
VVPFYSHSAETVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 40 - DNA, 32 bp, synthetic construct
  misc_feature 1..32: note = Synthetic polypeptide
  source 1..32: mol_type = other DNA
taatggaaacttcctcatgaaaaagtctttag

SEQ ID NO: 41 - DNA, 30 bp, synthetic construct
  misc_feature 1..30: note = Synthetic polypeptide
  source 1..30: mol_type = other DNA
acagagagaataacataaaaacagggaagc

SEQ ID NO: 42 - protein, 453 aa, synthetic construct
  REGION 1..453: note = Synthetic polypeptide
MIKIATRKYLGKQNVYDIGVERDHNFALKNGFIASNCFNVVPFYSHSAETVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 43 - DNA, 23 bp, synthetic construct
  misc_feature 1..23: note = Synthetic polypeptide
  source 1..23: mol_type = other DNA
tgcccctccctccctggcccagg

SEQ ID NO: 44 - DNA, 23 bp, synthetic construct
  misc_feature 1..23: note = Synthetic polypeptide
  source 1..23: mol_type = other DNA
ggaatcccttctgcagcacctgg

SEQ ID NO: 45 - DNA, 23 bp, synthetic construct
  misc_feature 1..23: note = Synthetic polypeptide
  source 1..23: mol_type = other DNA
gaacacaaagcatagactgcggg

SEQ ID NO: 46 - DNA, 23 bp, synthetic construct
  misc_feature 1..23: note = Synthetic polypeptide
  source 1..23: mol_type = other DNA
ggcccagactgagcacgtgatgg

SEQ ID NO: 47 - DNA, 23 bp, synthetic construct
  misc_feature 1..23: note = Synthetic polypeptide
  source 1..23: mol_type = other DNA
ggcactgcggctggaggtggggg

SEQ ID NO: 48 - DNA, 23 bp, synthetic construct
  misc_feature 1..23: note = Synthetic polypeptide
  source 1..23: mol_type = other DNA
gtcatcttagtcattacctgagg

(19) United States
(12) Patent Application Publication
(10) Pub. No.: US 2024/0209338 A1
(43) Pub. Date: Jun. 27, 2024

(54) EVOLUTION OF CYTIDINE DEAMINASES

(71) Applicants: President and Fellows of Harvard College, Cambridge, MA (US); The Broad Institute, Inc., Cambridge, MA (US)

(72) Inventors: David R. Liu, Cambridge, MA (US); Tina Wang, Cambridge, MA (US)

(73) Assignees: President and Fellows of Harvard College, Cambridge, MA (US); The Broad Institute, Inc., Cambridge, MA (US)

(21) Appl. No.: 18/419,414

(22) Filed: Jan. 22, 2024

Related U.S. Application Data
(62) Division of application No. 17/251,276, filed on Dec. 11, 2020, now Pat. No. 11,913,044.
(60) Provisional application No. 62/685,252, filed on Jun. 14, 2018.

(51) Int. Cl. C12N 9/78; C12N 9/22

(52) U.S. Cl. CPC C12N 9/78; C12N 9/22; C12Y 305/04005

(57) ABSTRACT

Some aspects of this disclosure relate to strategies, systems, methods, compositions, and kits that are useful for production (e.g., evolution) of cytidine deaminase protein variants that are characterized by increased soluble expression and/or stability relative to the wild-type cytidine deaminase protein from which they are evolved. In some embodiments, evolved cytidine deaminase variants described by the disclosure are useful for incorporation into targeted nucleic acid editing proteins, for example in fusion proteins with a Cas9 domain or variant thereof.

### RELATED APPLICATIONS

This application claims priority under 35 U.S.C. § 119(e) to U.S. Provisional Application, U.S. Ser. No. 62/685,252, filed Jun. 14, 2018, entitled “Evolution of Cytidine Deaminases,” which is incorporated herein by reference.

### GOVERNMENT SUPPORT

This invention was made with government support under grant numbers GM118062, GM119228, EB022376, and HG009490, awarded by the National Institutes of Health. The government has certain rights in the invention.

### BACKGROUND

Targeted editing of nucleic acid sequences, for example, the targeted cleavage or the targeted introduction of a specific modification into genomic DNA, is a highly promising approach for the study of gene function and also has the potential to provide new therapies for human genetic diseases. One drawback to the current technologies is that both NHEJ and HDR are stochastic processes that typically result in modest gene editing efficiencies as well as unwanted gene alterations that can compete with the desired alteration. Since many genetic diseases in principle can be treated by effecting a specific nucleotide change at a specific location in the genome (for example, a C to T change in a specific codon of a gene associated with a disease), the development of a programmable way to achieve such precision gene editing would represent both a powerful new research tool, as well as a potential new approach to gene editing-based human therapeutics.

Soluble protein expression is a desirable characteristic for the production and application of gene editing proteins. It is estimated that <50% of bacterial and <15% of non-bacterial proteins can be recombinantly expressed in soluble form in E. coli. However, many of these recombinantly expressed proteins either undergo proteolysis or are misfold into inclusion bodies. Moreover, engineering or evolving proteins towards improved or novel function can often lead to reduced soluble expression, impeding the development and application of proteins with tailor-made functional properties.

In addition to the challenge associated with simultaneously yet independently selecting for soluble expression and protein function, traditional directed evolution approaches to improving protein expression introduce additional drawbacks, including substantial time and effort requirements. Each round of traditional laboratory protein evolution can take a week or longer, rendering the prospect of evolving improvements in soluble expression in addition to new or improved functions an unattractive one. As a result, otherwise interesting targets for protein evolution are often not pursued due to the intractability of expression under standard laboratory conditions. A system that allows for the rapid improvement of protein expression, coupled with the preservation or improvement of protein function would therefore offer substantial benefits.

### SUMMARY

Cytidine deaminase proteins have been observed to be useful for correcting T to C mutations in genes associated with disease, for example, as described in International PCT Application No. PCT/US2016/058344, published on Apr. 27, 2017 as WO 2017/070632, the entire contents of which are incorporated herein by reference. However, certain cytidine deaminase proteins (e.g., apolipoprotein B mRNA editing catalytic subunit 1 (APOBEC1) proteins, such as rat APOBEC1) express poorly in E. coli and localize almost exclusively to the insoluble fraction, making recombinant production of such proteins difficult.

Some aspects of this disclosure relate to compositions (e.g., isolated nucleic acids, vectors, protein variants, etc.), and methods for improving physiochemical properties (e.g., thermostability, solubility, catalytic activity, etc.) of cytidine deaminase proteins using Phage-Assisted Continuous Evolution (PACE). Phage-assisted continuous evolution (PACE) can serve as a rapid, high-throughput system for evolving genes of interest. One advantage of the PACE technology is that both the time and human effort required to evolve a gene of interest are dramatically decreased as compared to conventional iterative evolution methods. During PACE, a phage vector carrying a gene encoding a gene of interest replicates in a flow of host cells through a fixed-volume vessel (a “lagoon”). For example, in some embodiments of PACE described herein, a population of bacteriophage vectors replicates in a continuous flow of bacterial host cells through the lagoon, wherein the flow rate of the host cells is adjusted so that the average time a host cell remains in the lagoon is shorter than the average time required for host cell division, but longer than the average life cycle of the vector, e.g., shorter than the average M13 bacteriophage life cycle. As a result, the population of vectors replicating in the lagoon can be varied by inducing mutations, and then enriching the population for desired variants by applying selective pressure, while the host cells do not effectively replicate in the lagoon.

Some aspects of this disclosure relate to systems for improving the stability and/or solubility of cytidine deaminase proteins evolved during PACE, and are referred to in some embodiments as evolution of cytidine deaminase proteins using Soluble Expression PACE (SE-PACE). The systems, including recombinant expression constructs, also referred to as vectors if they are in the form of a plasmid, can enhance selection of evolved proteins that are properly folded, have increased stability (e.g., thermodynamic stability), and/or solubility (e.g., enhanced soluble expression in bacteria, such as E. coli) while maintaining desired protein function. SE-PACE is generally described in U.S. Provisional Application, Ser. No. 62/559,919, filed Sep. 18, 2017, the entire contents of which are incorporated herein by reference. In some aspects, the disclosure relates to cytidine deaminase protein variants that are produced using SE-PACE.

In some aspects, the disclosure relates to a cytidine deaminase protein (e.g., cytidine deaminase protein variant) comprising an amino acid sequence that is at least 90% identical to a cytidine deaminase protein (for example an APOBEC protein, such as a rat APOBEC protein, rAPOBEC1, SEQ ID NO: 15), wherein the amino acid sequence includes at least one mutation occurring at a position selected from R33, G45, N57, N65, Y75, T101, F113, F113, A123, S149, A165, H166, T204, F205, and W224, or at a corresponding position in another cytidine deaminase.

In some embodiments, an amino acid sequence encoding a cytidine deaminase protein is at least 96%, at least 97%, at least 98%, or at least 99% identical to SEQ ID NO: 15

In some embodiments, a cytidine deaminase protein variant includes at least one of the following mutations: R33C, G45D, N57S, N65D, Y75H, T1011, F113C, F113R, A123E, S149Y, A165T, H166N, T204P, F205S, or W224R.

In some embodiments, a cytidine deaminase protein variant does not comprise a mutation at position E63.

In some embodiments, the amino acid sequence of a cytidine deaminase protein variant includes mutations at positions F113, A123, and F205. In some embodiments, the amino acid sequence of a cytidine deaminase protein variant includes mutations at positions F113C, A123E, and F205S. In some embodiments, the amino acid sequence of a cytidine deaminase protein variant is represented by SEQ ID NO: 16.

In some embodiments, the amino acid sequence of a cytidine deaminase protein variant includes mutations at positions G45, Y75, F113, A123, H166, and F205. In some embodiments, the amino acid sequence of a cytidine deaminase protein variant includes mutations at positions G45D, Y75H, F113C, A123E, H166N, and F205S. In some embodiments, the amino acid sequence of a cytidine deaminase protein variant is represented by SEQ ID NO: 17.

In some embodiments, the amino acid sequence of a cytidine deaminase protein variant includes mutations at positions F113, A123, A165, and F205. In some embodiments, the amino acid sequence of a cytidine deaminase protein variant includes mutations at positions F113C, A123E, A165T, and F205S. In some embodiments, the amino acid sequence of a cytidine deaminase protein variant is represented by SEQ ID NO: 19.

In some aspects, the disclosure relates to an isolated nucleic acid encoding a cytidine deaminase protein variant as described by the disclosure. In some embodiments, an isolated nucleic acid comprises or consists of the sequence set forth in any one of SEQ ID NOs: 7, 8, or 10. In some embodiments, an isolated nucleic acid comprises or consists of a sequence that is at least 70%, at least 80%, at least 90%, at least 95%, or at least 99% identical to the sequence set forth in any one of SEQ ID NOs: 7, 8, or 10.

In some embodiments, an isolated nucleic acid is codon optimized for expression (e.g., optimized for expression in E. coli cells or mammalian cells). In some embodiments, and isolated nucleic acid comprises or consists of the sequence set forth in any one of SEQ ID NOS: 12-14. In some embodiments, an isolated nucleic acid comprises or consists of a sequence that is at least 70%, at least 80%, at least 90%, at least 95%, or at least 99% identical to the sequence set forth in any one of SEQ ID NOs: 12-14.

In some aspects, the disclosure relates to a host cell comprising a cytidine deaminase protein variant as described by the disclosure. In some aspects, the disclosure relates to an isolated nucleic acid as described by the disclosure. In some embodiments, a host cell is a bacterial cell. In some aspects, a host cell is a mammalian cell. In some embodiments, a bacterial cell is an E. coli cell. In some embodiments, a mammalian cell is a human cell.

In some aspects, the disclosure relates to a fusion protein comprising: (i) a RNA-programmable nuclease (e.g., a Cas9 domain); and (ii) a cytosine deaminase protein variant as described by the disclosure. In some embodiments, a fusion protein further comprises (iii) a uracil glycosylase inhibitor (UGI) domain.

In some embodiments, a RNA-programmable nuclease (e.g. a Cas9 domain) comprises an amino acid sequence that is at least 85% identical to the amino acid sequence provided in SEQ ID NO: 20.

In some embodiments, a Cas9 domain is a Cas9 nickase domain that cuts a nucleotide target strand of a nucleotide duplex, wherein the nucleotide target strand is the strand that binds to a gRNA of the Cas9 nickase domain, for example as provided in SEQ ID NO: 21.

In some embodiments, a UGI domain comprises a domain capable of inhibiting Uracil DNA Glycosylase (UDG) activity. In some embodiments, a UGI domain comprises an amino acid sequence that is at least 85% identical to SEQ ID NO: 22.

In some embodiments, fusion proteins provided herein further comprise a nuclear localization sequence (NLS).

In some aspects, the disclosure relates to a vector system comprising: (i) a selection phagemid comprising an isolated nucleic acid comprising an expression construct encoding a fusion protein comprising, in the following order: a GCN4 peptide, a cytidine deaminase protein, a T7 RNA polymerase, and an E. coli RNA polymerase omega subunit (rpoZ); (ii) a first accessory plasmid comprising an isolated nucleic acid comprising an expression construct encoding a RNA polymerase I promoter operably linked to an expression cassette comprising, in the following order: a sequence encoding a M13 phage gIII protein signal peptide, and a sequence encoding a Npu split intein N-terminal portion, wherein the sequence encoding the gIII protein signal peptide lacks one or more nucleic acid bases of the signal peptide domain; and (iii) a second accessory plasmid comprising an isolated nucleic acid comprising an expression construct encoding a T7 promoter operably linked to an expression cassette comprising, in the following order: a sequence encoding a Npu split intein C-terminal portion, and a sequence encoding a M13 phage gIII protein, wherein the sequence encoding the gIII protein lacks one or more nucleic acid bases in the signal peptide domain.

In some embodiments, a vector system as described by the disclosure further comprises a mutagenesis plasmid.

In some aspects, the disclosure relates to a kit comprising: a first container housing a selection phagemid as described by the disclosure; a second container housing a first accessory plasmid as described by the disclosure; and a third container housing a second accessory plasmid as described by the disclosure.

In some embodiments, a kit further comprises a container housing one or more bacterial cells. In some embodiments, the bacterial cells are E. coli cells.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIGS. 1A-1B show a schematic depicting cheat-resistant, activity-independent selection for protein expression (e.g., activity independent SE-PACE). FIG. 1A is a schematic depicting a protein of interest (POI) fused between an affinity tag (e.g., a GCN4 peptide) and T7n-rpoZ, allowing both the protein expression and protein binding selections to operate. As the selection phage (SP) carries the GCN4 peptide linked to rpoZ, they begin PACE with the ability to pass the protein binding selection. FIG. 1B is a schematic showing that any selection phage (SP) that cheat by forming truncations in the POI lose the ability to propagate on the protein binding selection, as the GCN4 peptide is no longer connected to rpoZ. Therefore, these phage are expected to wash out of the lagoon.

FIGS. 2A-2C show activity independent SE-PACE of rAPBOEC1. FIG. 2A shows representative data for expression of rAPOBEC and rAPOBEC variants (36.1, 43.1, 43.2rev) in BL21 DE3 cells at 37 ° C. FIG. 2B shows consensus mutations from SP isolated from lagoons 1 and 2 of first two PACE experiments. FIG. 2C shows mutations in rAPOBEC1 and rAPOBEC1 variants after 370 h of PACE.

FIGS. 3A-3D show activity-independent selection for rAPOBEC1 protein variants in SE-PACE. FIG. 3A shows representative plaque forming unit (pfu) data from activity independent SE-PACE of cytidine deaminase rAPOBEC1. The stringency of the protein expression selection was modulated through increasing the rate of lagoon dilution (flow rate), as well as the use of activity-diminishing T7 RNAP and T7 promoter mutants in the accessory plasmid (AP). FIG. 3B shows rAPOBEC1 variants obtained after 186 h and 370 h show greatly improved soluble expression at 37° C. when compared to wild-type rAPOBEC1. FIG. 3C shows that Base Editor (BE2) variants employing evolved rAPOBEC1 variants have enhanced apparent activity in E. coli, as measured by cells surviving selection on increasing concentrations of chloramphenicol due to BE2-dependent reversion of an active site mutation in chloramphenicol acetyl transferase. Data reflects the mean and s.d. of three technical replicates (unique clones). FIG. 3D shows data indicating that Base Editor (BE3) variants that include evolved rAPOBEC1 variants 36.1 and 43.1 show enhanced editing efficiency in HEK293T cells. Data reflects the mean and s.e.m. of four biological replicates (experiments performed on different days).

FIGS. 4A-4B show activity of rAPOBEC1 variants in BL21 cells. FIG. 4A shows rAPOBEC1 variants from PACE retain deaminase activity in E. coli, as measured by cells surviving selection on rifampin. FIG. 4B shows total colony forming units (cfu) from overnight cultures of BL21 cells after induction of rAPOBEC1 variants. Data reflect the mean and s.e.m. of six biological replicates.

FIG. 5 shows product purity of BE3 mediated cytidine editing in HEK293T cells using wild-type rAPOBEC1 and evolved variants. The top panel refers to editing at the bolded C at each site. The bottom panel shows indel formation resulting from editing with BE3 variants in HEK293T cells at the same sites shown in the top panel. Sequences correspond from top to bottom to SEQ ID NOs: 43-48.

### DEFINITIONS

The term “phage-assisted continuous evolution (PACE),” as used herein, refers to continuous evolution that employs phage as viral vectors. The general concept of PACE technology has been described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Application, U.S. Pat. No. 9,023,594, issued May 5, 2015, International PCT Application, PCT/US2015/012022, filed Jan. 20, 2015, published as WO 2015/134121 on Sep. 11, 2015, and International PCT Application, PCT/US2016/027795, filed Apr. 15, 2016, published as WO 2016/168631 on Oct. 20, 2016, the entire contents of each of which are incorporated herein by reference.

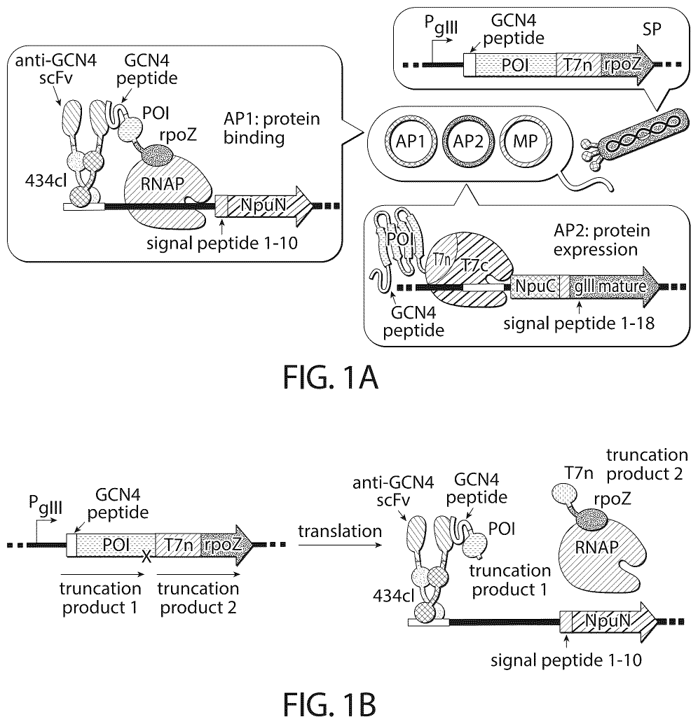

The term “soluble expression phage-assisted continuous evolution (SE-PACE),” as used herein, refers to continuous evolution that employs a PACE-compatible “AND” gate that uses a split-intein pIII, which renders T7 RNA polymerase (T7 RNAP) activity dependent on the soluble expression of a target protein, and also uses the resulting T7 RNAP activity to drive gene III expression. In some embodiments, SE-PACE allows for two positive selections to take place in the same PACE experiment. The general concept of SE-PACE technology has been described, for example, in U.S. Provisional Application, Ser. No. 62/559,919, filed Sep. 18, 2017, the entire contents of which are incorporated herein by reference. One embodiment of a SE-PACE system is described in FIG. 1A.

The term “continuous evolution,” as used herein, refers to an evolution process, in which a population of nucleic acids encoding a gene to be evolved is subjected to multiple rounds of (a) replication, (b) mutation, and (c) selection to produce a desired evolved version of the gene to be evolved that is different from the original version of the gene, for example, in that a gene product, such as, e.g., an RNA or protein encoded by the gene, exhibits a new activity not present in the original version of the gene product, or in that an activity of a gene product encoded by the original gene to be evolved is modulated (increased or decreased). The multiple rounds can be performed without investigator intervention, and the steps (a)-(c) can be carried out simultaneously. Typically, the evolution procedure is carried out in vitro, for example, using cells in culture as host cells. In general, a continuous evolution process provided herein relies on a system in which a gene encoding a gene product of interest is provided in a nucleic acid vector that undergoes a life-cycle including replication in a host cell and transfer to another host cell, wherein a critical component of the life-cycle is deactivated and reactivation of the component is dependent upon an activity of the gene to be evolved that is a result of a mutation in the nucleic acid vector.

The term “vector,” as used herein, refers to a nucleic acid that can be modified to encode a gene of interest and that is able to enter into a host cell, mutate and replicate within the host cell, and then transfer a replicated form of the vector into another host cell. Exemplary suitable vectors include viral vectors, such as retroviral vectors or bacteriophages, and conjugative plasmids. Additional suitable vectors will be apparent to those of skill in the art based on the instant disclosure.

The term “viral vector,” as used herein, refers to a nucleic acid comprising a viral genome that, when introduced into a suitable host cell, can be replicated and packaged into viral particles able to transfer the viral genome into another host cell. The term viral vector extends to vectors comprising truncated or partial viral genomes. For example, in some embodiments, a viral vector is provided that lacks a gene encoding a protein essential for the generation of infectious viral particles. In suitable host cells, for example, host cells comprising the lacking gene under the control of a conditional promoter, however, such truncated viral vectors can replicate and generate viral particles able to transfer the truncated viral genome into another host cell. In some embodiments, the viral vector is a phage, for example, a filamentous phage (e.g., an M13 phage). In some embodiments, a viral vector, for example, a phage vector, is provided that comprises a gene of interest to be evolved.

The term “phage,” as used herein interchangeably with the term “bacteriophage,” refers to a virus that infects bacterial cells. Typically, phages consist of an outer protein capsid enclosing genetic material. The genetic material can be ssRNA, dsRNA, ssDNA, or dsDNA, in either linear or circular form. Phages and phage vectors are well known to those of skill in the art and non-limiting examples of phages that are useful for carrying out the methods provided herein are λ (Lysogen), T2, T4, T7, T12, R17, M13, MS2, G4, P1, P2, P4, Phi X174, N4, Φ6, and Φ29. In certain embodiments, the phage utilized in the present invention is M13. Additional suitable phages and host cells will be apparent to those of skill in the art and the invention is not limited in this aspect. For an exemplary description of additional suitable phages and host cells, see Elizabeth Kutter and Alexander Sulakvelidze: Bacteriophages: Biology and Applications. CRC Press; 1^{st} edition (December 2004), ISBN: 0849313368; Martha R. J. Clokie and Andrew M. Kropinski: Bacteriophages: Methods and Protocols, Volume 1: Isolation, Characterization, and Interactions (Methods in Molecular Biology) Humana Press; 1^{st} edition (December, 2008), ISBN: 1588296822; Martha R. J. Clokie and Andrew M. Kropinski: Bacteriophages: Methods and Protocols, Volume 2: Molecular and Applied Aspects (Methods in Molecular Biology) Humana Press; 1^{st} edition (December 2008), ISBN: 1603275649; all of which are incorporated herein in their entirety by reference for disclosure of suitable phages and host cells as well as methods and protocols for isolation, culture, and manipulation of such phages).

The term “accessory plasmid,” as used herein, refers to a plasmid comprising a gene required for the generation of infectious viral particles under the control of a conditional promoter. In the context of continuous evolution of genes, transcription from the conditional promoter of the accessory plasmid is typically activated, directly or indirectly, by a function of the gene to be evolved. Accordingly, the accessory plasmid serves the function of conveying a competitive advantage to those viral vectors in a given population of viral vectors that carry a version of the gene to be evolved able to activate the conditional promoter or able to activate the conditional promoter more strongly than other versions of the gene to be evolved. In some embodiments, only viral vectors carrying an “activating” version of the gene to be evolved will be able to induce expression of the gene required to generate infectious viral particles in the host cell, and, thus, allow for packaging and propagation of the viral genome in the flow of host cells. Vectors carrying non-activating versions of the gene to be evolved, on the other hand, will not induce expression of the gene required to generate infectious viral vectors, and, thus, will not be packaged into viral particles that can infect fresh host cells.

The term “helper phage,” as used herein, interchangeable with the terms “helper phagemid” and “helper plasmid,” refers to a nucleic acid construct comprising a phage gene required for the phage life cycle, or a plurality of such genes, but lacking a structural element required for genome packaging into a phage particle. For example, a helper phage may provide a wild-type phage genome lacking a phage origin of replication. In some embodiments, a helper phage is provided that comprises a gene required for the generation of phage particles, but lacks a gene required for the generation of infectious particles, for example, a full-length pIII gene. In some embodiments, the helper phage provides only some, but not all, genes for the generation of infectious phage particles. Helper phages are useful to allow modified phages that lack a gene for the generation of infectious phage particles to complete the phage life cycle in a host cell. Typically, a helper phage will comprise the genes for the generation of infectious phage particles that are lacking in the phage genome, thus complementing the phage genome. In the continuous evolution context, the helper phage typically complements the selection phage, but both lack a phage gene required for the production of infectious phage particles.

The term “selection phage,” as used herein interchangeably with the term “selection plasmid,” refers to a modified phage that comprises a nucleic acid sequence encoding a cytidine deaminase to be evolved, and lacks a full-length gene encoding a protein required for the generation of infectious phage particles. For example, some M13 selection phages provided herein comprise a nucleic acid sequence encoding a gene to be evolved, e.g., under the control of an M13 promoter, and lack all or part of a phage gene encoding a protein required for the generation of infectious phage particles, e.g., gI, gII, gIII, gIV, gV, gVI, gVII, gVIII, gIX, or gX, or any combination thereof. For example, some M13 selection phages provided herein comprise a nucleic acid sequence encoding a cytidine deaminase protein to be evolved, e.g., under the control of an M13 promoter, and lack all or part of a gene encoding a protein required for the generation of infective phage particles, e.g., the gIII gene encoding the pIII protein.

The term “mutagenesis plasmid,” as used herein, refers to a plasmid comprising a gene encoding a gene product that acts as a mutagen. In some embodiments, the gene encodes a DNA polymerase lacking a proofreading capability. In some embodiments, the gene is a gene involved in the bacterial SOS stress response, for example, a UmuC, UmuD′, or RecA gene. In some embodiments, the gene is a GATC methylase gene, for example, a deoxyadenosine methylase (dam methylase) gene. In some embodiments, the gene is involved in binding of hemimethylated GATC sequences, for example, a seqA gene. In some embodiments, the gene is involved with repression of mutagenic nucleobase export, for example emrR. In some embodiments, the gene is involved with inhibition of uracil DNA-glycosylase, for example a Uracil Glycosylase Inhibitor (ugi) gene. In some embodiments, the gene is involved with deamination of cytidine (e.g., a cytidine deaminase from Petromyzon marinus), for example, cytidine deaminase 1 (CDA1). Mutagenesis plasmids (also referred to as mutagenesis constructs) are described, for example by International Patent Application, PCT/US2016/027795, filed Apr. 16, 2016, published as WO2016/168631 on Oct. 20, 2016, the entire contents of which are incorporated herein by reference.

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′-N-phosphoramidite linkages).

The term “protein,” as used herein, refers to a polymer of amino acid residues linked together by peptide bonds. The term, as used herein, refers to proteins, polypeptides, and peptide of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain; see, for example, cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive protein may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein may also be a single molecule or may be a multi-molecular complex. A protein may be just a fragment of a naturally occurring protein or peptide. A protein may be naturally occurring, recombinant, or synthetic, or any combination of these.

The term “evolved deaminase protein,” as used herein, refers to a cytidine deaminase protein variant that is expressed by a gene of interest (e.g., a gene encoding a cytidine deaminase protein) that has been subjected to continuous evolution, such as PACE or SE-PACE.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The term “deaminase”, “deaminase protein”, or “deaminase domain,” as used herein, refers to a protein or enzyme that catalyzes a deamination reaction. In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytosine deaminase, catalyzing the hydrolytic deamination of cytosine to uracil. In some embodiments, the deaminase or deaminase domain is a naturally-occurring deaminase from an organism, such as a human, chimpanzee, gorilla, monkey, cow, dog, rat, or mouse.

The term “cytidine deaminase protein variant,” as used herein, refers to a cytidine deaminase protein having one or more amino acid variations introduced into the amino acid sequence, e.g., as a result of application of the PACE method or SE-PACE method, as compared to the amino acid sequence of a naturally-occurring or wild-type cytidine deaminase protein. Amino acid sequence variations may include one or more mutated residues within the amino acid sequence of the cytidine deaminase protein variant, e.g., as a result of a change in the nucleotide sequence encoding the protein that results in a change in the codon at any particular position in the coding sequence, the deletion of one or more amino acids (e.g., a truncated protein), the insertion of one or more amino acids, or any combination of the foregoing. In some embodiments, the deaminase or deaminase domain variant is a variant of a naturally-occurring deaminase from an organism, that does not occur in nature. In some embodiments, a deaminase variant or deaminase domain variant is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

A “wild-type cytidine deaminase protein” refers to the amino acid sequence of a cytidine deaminase protein as it naturally occurs in the genome of the host from which it is derived. Examples of a wild-type cytidine deaminase proteins include rat apolipoprotein B mRNA editing catalytic subunit 1 (rAPOBEC1), which is represented by the amino acid sequence set forth in SEQ ID NO: 15, and human APOBEC1, which is represented by the amino acid sequence of NCBI Accession Number NP_001291495.1.

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “RNA-programmable nuclease,” and “RNA-guided nuclease” are used interchangeably herein and refer to a nuclease that forms a complex with (e.g., binds or associates with) one or more RNA that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. For example, in some embodiments, domain (2) is identical or homologous to a tracrRNA as provided in Jinek et al., Science 337:816-821(2012), the entire contents of which is incorporated herein by reference. Other examples of gRNAs (e.g., those including domain 2) can be found in U.S. Provisional Patent Application, U.S. Ser. No. 61/874,682, filed Sep. 6, 2013, entitled “Switchable Cas9 Nucleases And Uses Thereof,” and U.S. Provisional Patent Application, U.S. Ser. No. 61/874,746, filed Sep. 6, 2013, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J.J., McShan W.M., Ajdic D.J., Savic D.J., Savic G., Lyon K., Primeaux C., Sezate S., Suvorov A.N., Kenton S., Lai H.S., Lin S.P., Qian Y., Jia H.G., Najar F.Z., Ren Q., Zhu H., Song L., White J., Yuan X., Clifton S.W., Roc B.A., Mclaughlin R.E., Proc. Natl. Acad. Sci. U.S.A. 98:4658-4663(2001); “CRISPR RNA maturation by trans-encoded small RNA and host factor RNase III.” Deltcheva E., Chylinski K., Sharma C.M., Gonzales K., Chao Y., Pirzada Z.A., Eckert M.R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J.A., Charpentier E. Science 337:816-821(2012), the entire contents of each of which are incorporated herein by reference.

Because RNA-programmable nucleases (e.g., Cas9) use RNA:DNA hybridization to target DNA cleavage sites, these proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W.Y. et al. Efficient genome editing in zebrafish using a CRISPR-Cas system. Nature biotechnology 31, 227-229 (2013); Jinek, M. et al. RNA-programmed genome editing in human cells. eLife 2, e00471 (2013); Dicarlo, J.E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013); the entire contents of each of which are incorporated herein by reference).

The term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J.A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J.J., McShan W.M., Ajdic D.J., Savic D.J., Savic G., Lyon K., Primeaux C., Sezate S., Suvorov A.N., Kenton S., Lai H.S., Lin S.P., Qian Y., Jia H.G., Najar F.Z., Ren Q., Zhu H., Song L., White J., Yuan X., Clifton S.W., Roc B.A., Mclaughlin R.E., Proc. Natl. Acad. Sci. U.S.A. 98:4658-4663(2001); “CRISPR RNA maturation by trans-encoded small RNA and host factor RNase III.” Deltcheva E., Chylinski K., Sharma C.M., Gonzales K., Chao Y., Pirzada Z.A., Eckert M.R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J.A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase.

A nuclease-inactivated Cas9 protein may interchangeably be referred to as a “dCas9” protein (for nuclease-“dead” Cas9). Methods for generating a Cas9 protein (or a fragment thereof) having an inactive DNA cleavage domain are known (Sec, e.g., Jinek et al., Science. 337:816-821(2012); Qi et al., “Repurposing CRISPR as an RNA-Guided Platform for Sequence-Specific Control of Gene Expression” (2013) Cell. 28; 152(5): 1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28;152(5):1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, or more amino acid changes compared to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of wild type Cas9. In some embodiments, wild type Cas9 corresponds to Cas9 from Streptococcus pyogenes (NCBI Reference Sequence: NC_017053.1, SEQ ID NO: 23 (nucleotide); SEQ ID NO 24 (amino acid)).

(SEQ ID NO: 23)
ATGGATAAGAAATACTCAATAGGCTTAGATATCGGCACAAATAGCGTCGG
ATGGGCGGTGATCACTGATGATTATAAGGTTCCGTCTAAAAAGTTCAAGG
TTCTGGGAAATACAGACCGCCACAGTATCAAAAAAAATCTTATAGGGGCT
CTTTTATTTGGCAGTGGAGAGACAGCGGAAGCGACTCGTCTCAAACGGAC
AGCTCGTAGAAGGTATACACGTCGGAAGAATCGTATTTGTTATCTACAGG
AGATTTTTTCAAATGAGATGGCGAAAGTAGATGATAGTTTCTTTCATCGA
CTTGAAGAGTCTTTTTTGGTGGAAGAAGACAAGAAGCATGAACGTCATCC
TATTTTTGGAAATATAGTAGATGAAGTTGCTTATCATGAGAAATATCCAA
CTATCTATCATCTGCGAAAAAAATTGGCAGATTCTACTGATAAAGCGGAT
TTGCGCTTAATCTATTTGGCCTTAGCGCATATGATTAAGTTTCGTGGTCA
TTTTTTGATTGAGGGAGATTTAAATCCTGATAATAGTGATGTGGACAAAC
TATTTATCCAGTTGGTACAAATCTACAATCAATTATTTGAAGAAAACCCT
ATTAACGCAAGTAGAGTAGATGCTAAAGCGATTCTTTCTGCACGATTGAG
TAAATCAAGACGATTAGAAAATCTCATTGCTCAGCTCCCCGGTGAGAAGA
GAAATGGCTTGTTTGGGAATCTCATTGCTTTGTCATTGGGATTGACCCCT
AATTTTAAATCAAATTTTGATTTGGCAGAAGATGCTAAATTACAGCTTTC
AAAAGATACTTACGATGATGATTTAGATAATTTATTGGCGCAAATTGGAG
ATCAATATGCTGATTTGTTTTTGGCAGCTAAGAATTTATCAGATGCTATT
TTACTTTCAGATATCCTAAGAGTAAATAGTGAAATAACTAAGGCTCCCCT
ATCAGCTTCAATGATTAAGCGCTACGATGAACATCATCAAGACTTGACTC
TTTTAAAAGCTTTAGTTCGACAACAACTTCCAGAAAAGTATAAAGAAATC
TTTTTTGATCAATCAAAAAACGGATATGCAGGTTATATTGATGGGGGAGC
TAGCCAAGAAGAATTTTATAAATTTATCAAACCAATTTTAGAAAAAATGG
ATGGTACTGAGGAATTATTGGTGAAACTAAATCGTGAAGATTTGCTGCGC
AAGCAACGGACCTTTGACAACGGCTCTATTCCCCATCAAATTCACTTGGG
TGAGCTGCATGCTATTTTGAGAAGACAAGAAGACTTTTATCCATTTTTAA
AAGACAATCGTGAGAAGATTGAAAAAATCTTGACTTTTCGAATTCCTTAT
TATGTTGGTCCATTGGCGCGTGGCAATAGTCGTTTTGCATGGATGACTCG
GAAGTCTGAAGAAACAATTACCCCATGGAATTTTGAAGAAGTTGTCGATA
AAGGTGCTTCAGCTCAATCATTTATTGAACGCATGACAAACTTTGATAAA
AATCTTCCAAATGAAAAAGTACTACCAAAACATAGTTTGCTTTATGAGTA
TTTTACGGTTTATAACGAATTGACAAAGGTCAAATATGTTACTGAGGGAA
TGCGAAAACCAGCATTTCTTTCAGGTGAACAGAAGAAAGCCATTGTTGAT
TTACTCTTCAAAACAAATCGAAAAGTAACCGTTAAGCAATTAAAAGAAGA
TTATTTCAAAAAAATAGAATGTTTTGATAGTGTTGAAATTTCAGGAGTTG
AAGATAGATTTAATGCTTCATTAGGCGCCTACCATGATTTGCTAAAAATT
ATTAAAGATAAAGATTTTTTGGATAATGAAGAAAATGAAGATATCTTAGA
GGATATTGTTTTAACATTGACCTTATTTGAAGATAGGGGGATGATTGAGG
AAAGACTTAAAACATATGCTCACCTCTTTGATGATAAGGTGATGAAACAG
CTTAAACGTCGCCGTTATACTGGTTGGGGACGTTTGTCTCGAAAATTGAT
TAATGGTATTAGGGATAAGCAATCTGGCAAAACAATATTAGATTTTTTGA
AATCAGATGGTTTTGCCAATCGCAATTTTATGCAGCTGATCCATGATGAT
AGTTTGACATTTAAAGAAGATATTCAAAAAGCACAGGTGTCTGGACAAGG
CCATAGTTTACATGAACAGATTGCTAACTTAGCTGGCAGTCCTGCTATTA
AAAAAGGTATTTTACAGACTGTAAAAATTGTTGATGAACTGGTCAAAGTA
ATGGGGCATAAGCCAGAAAATATCGTTATTGAAATGGCACGTGAAAATCA
GACAACTCAAAAGGGCCAGAAAAATTCGCGAGAGCGTATGAAACGAATCG
AAGAAGGTATCAAAGAATTAGGAAGTCAGATTCTTAAAGAGCATCCTGTT
GAAAATACTCAATTGCAAAATGAAAAGCTCTATCTCTATTATCTACAAAA
TGGAAGAGACATGTATGTGGACCAAGAATTAGATATTAATCGTTTAAGTG
ATTATGATGTCGATCACATTGTTCCACAAAGTTTCATTAAAGACGATTCA
ATAGACAATAAGGTACTAACGCGTTCTGATAAAAATCGTGGTAAATCGGA
TAACGTTCCAAGTGAAGAAGTAGTCAAAAAGATGAAAAACTATTGGAGAC
AACTTCTAAACGCCAAGTTAATCACTCAACGTAAGTTTGATAATTTAACG
AAAGCTGAACGTGGAGGTTTGAGTGAACTTGATAAAGCTGGTTTTATCAA
ACGCCAATTGGTTGAAACTCGCCAAATCACTAAGCATGTGGCACAAATTT
TGGATAGTCGCATGAATACTAAATACGATGAAAATGATAAACTTATTCGA
GAGGTTAAAGTGATTACCTTAAAATCTAAATTAGTTTCTGACTTCCGAAA
AGATTTCCAATTCTATAAAGTACGTGAGATTAACAATTACCATCATGCCC
ATGATGCGTATCTAAATGCCGTCGTTGGAACTGCTTTGATTAAGAAATAT
CCAAAACTTGAATCGGAGTTTGTCTATGGTGATTATAAAGTTTATGATGT
TCGTAAAATGATTGCTAAGTCTGAGCAAGAAATAGGCAAAGCAACCGCAA
AATATTTCTTTTACTCTAATATCATGAACTTCTTCAAAACAGAAATTACA
CTTGCAAATGGAGAGATTCGCAAACGCCCTCTAATCGAAACTAATGGGGA
AACTGGAGAAATTGTCTGGGATAAAGGGCGAGATTTTGCCACAGTGCGCA
AAGTATTGTCCATGCCCCAAGTCAATATTGTCAAGAAAACAGAAGTACAG
ACAGGCGGATTCTCCAAGGAGTCAATTTTACCAAAAAGAAATTCGGACAA
GCTTATTGCTCGTAAAAAAGACTGGGATCCAAAAAAATATGGTGGTTTTG
ATAGTCCAACGGTAGCTTATTCAGTCCTAGTGGTTGCTAAGGTGGAAAAA
GGGAAATCGAAGAAGTTAAAATCCGTTAAAGAGTTACTAGGGATCACAAT
TATGGAAAGAAGTTCCTTTGAAAAAAATCCGATTGACTTTTTAGAAGCTA
AAGGATATAAGGAAGTTAAAAAAGACTTAATCATTAAACTACCTAAATAT
AGTCTTTTTGAGTTAGAAAACGGTCGTAAACGGATGCTGGCTAGTGCCGG
AGAATTACAAAAAGGAAATGAGCTGGCTCTGCCAAGCAAATATGTGAATT
TTTTATATTTAGCTAGTCATTATGAAAAGTTGAAGGGTAGTCCAGAAGAT
AACGAACAAAAACAATTGTTTGTGGAGCAGCATAAGCATTATTTAGATGA
GATTATTGAGCAAATCAGTGAATTTTCTAAGCGTGTTATTTTAGCAGATG
CCAATTTAGATAAAGTTCTTAGTGCATATAACAAACATAGAGACAAACCA
ATACGTGAACAAGCAGAAAATATTATTCATTTATTTACGTTGACGAATCT
TGGAGCTCCCGCTGCTTTTAAATATTTTGATACAACAATTGATCGTAAAC
GATATACGTCTACAAAAGAAGTTTTAGATGCCACTCTTATCCATCAATCC
ATCACTGGTCTTTATGAAACACGCATTGATTTGAGTCAGCTAGGAGGTGA
CTGA

(SEQ ID NO: 24)
MDKKYSIGLDIGTNSVGWAVITDDYKVPSKKFKVLGNTDRHSIKKNLIGA
LLFGSGETAEATRLKRTARRRYTRRKNRICYLQEIFSNEMAKVDDSFFHR
LEESFLVEEDKKHERHPIFGNIVDEVAYHEKYPTIYHLRKKLADSTDKAD
LRLIYLALAHMIKFRGHFLIEGDLNPDNSDVDKLFIQLVQIYNQLFEENP
INASRVDAKAILSARLSKSRRLENLIAQLPGEKRNGLFGNLIALSLGLTP
NFKSNFDLAEDAKLQLSKDTYDDDLDNLLAQIGDQYADLFLAAKNLSDAI
LLSDILRVNSEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGAYHDLLKI
IKDKDFLDNEENEDILEDIVLTLTLFEDRGMIEERLKTYAHLFDDKVMKQ
LKRRRYTGWGRLSRKLINGIRDKQSGKTILDFLKSDGFANRNFMQLIHDD
SLTFKEDIQKAQVSGQGHSLHEQIANLAGSPAIKKGILQTVKIVDELVKV
MGHKPENIVIEMARENQTTQKGQKNSRERMKRIEEGIKELGSQILKEHPV
ENTQLQNEKLYLYYLQNGRDMYVDQELDINRLSDYDVDHIVPQSFIKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD
(single underline: HNH domain; double underline:
RuvC domain)

In some embodiments, wild type Cas9 corresponds to, or comprises SEQ ID NO: 25 (nucleotide) and/or SEQ ID NO: 26 (amino acid):

(SEQ ID NO: 25)
ATGGATAAAAAGTATTCTATTGGTTTAGACATCGGCACTAATTCCGTTGG
ATGGGCTGTCATAACCGATGAATACAAAGTACCTTCAAAGAAATTTAAGG
TGTTGGGGAACACAGACCGTCATTCGATTAAAAAGAATCTTATCGGTGCC
CTCCTATTCGATAGTGGCGAAACGGCAGAGGCGACTCGCCTGAAACGAAC
CGCTCGGAGAAGGTATACACGTCGCAAGAACCGAATATGTTACTTACAAG
AAATTTTTAGCAATGAGATGGCCAAAGTTGACGATTCTTTCTTTCACCGT
TTGGAAGAGTCCTTCCTTGTCGAAGAGGACAAGAAACATGAACGGCACCC
CATCTTTGGAAACATAGTAGATGAGGTGGCATATCATGAAAAGTACCCAA
CGATTTATCACCTCAGAAAAAAGCTAGTTGACTCAACTGATAAAGCGGAC
CTGAGGTTAATCTACTTGGCTCTTGCCCATATGATAAAGTTCCGTGGGCA
CTTTCTCATTGAGGGTGATCTAAATCCGGACAACTCGGATGTCGACAAAC
TGTTCATCCAGTTAGTACAAACCTATAATCAGTTGTTTGAAGAGAACCCT
ATAAATGCAAGTGGCGTGGATGCGAAGGCTATTCTTAGCGCCCGCCTCTC
TAAATCCCGACGGCTAGAAAACCTGATCGCACAATTACCCGGAGAGAAGA
AAAATGGGTTGTTCGGTAACCTTATAGCGCTCTCACTAGGCCTGACACCA
AATTTTAAGTCGAACTTCGACTTAGCTGAAGATGCCAAATTGCAGCTTAG
TAAGGACACGTACGATGACGATCTCGACAATCTACTGGCACAAATTGGAG
ATCAGTATGCGGACTTATTTTTGGCTGCCAAAAACCTTAGCGATGCAATC
CTCCTATCTGACATACTGAGAGTTAATACTGAGATTACCAAGGCGCCGTT
ATCCGCTTCAATGATCAAAAGGTACGATGAACATCACCAAGACTTGACAC
TTCTCAAGGCCCTAGTCCGTCAGCAACTGCCTGAGAAATATAAGGAAATA
TTCTTTGATCAGTCGAAAAACGGGTACGCAGGTTATATTGACGGCGGAGC
GAGTCAAGAGGAATTCTACAAGTTTATCAAACCCATATTAGAGAAGATGG
ATGGGACGGAAGAGTTGCTTGTAAAACTCAATCGCGAAGATCTACTGCGA
AAGCAGCGGACTTTCGACAACGGTAGCATTCCACATCAAATCCACTTAGG
CGAATTGCATGCTATACTTAGAAGGCAGGAGGATTTTTATCCGTTCCTCA
AAGACAATCGTGAAAAGATTGAGAAAATCCTAACCTTTCGCATACCTTAC
TATGTGGGACCCCTGGCCCGAGGGAACTCTCGGTTCGCATGGATGACAAG
AAAGTCCGAAGAAACGATTACTCCATGGAATTTTGAGGAAGTTGTCGATA
AAGGTGCGTCAGCTCAATCGTTCATCGAGAGGATGACCAACTTTGACAAG
AATTTACCGAACGAAAAAGTATTGCCTAAGCACAGTTTACTTTACGAGTA
TTTCACAGTGTACAATGAACTCACGAAAGTTAAGTATGTCACTGAGGGCA
TGCGTAAACCCGCCTTTCTAAGCGGAGAACAGAAGAAAGCAATAGTAGAT
CTGTTATTCAAGACCAACCGCAAAGTGACAGTTAAGCAATTGAAAGAGGA
CTACTTTAAGAAAATTGAATGCTTCGATTCTGTCGAGATCTCCGGGGTAG
AAGATCGATTTAATGCGTCACTTGGTACGTATCATGACCTCCTAAAGATA
ATTAAAGATAAGGACTTCCTGGATAACGAAGAGAATGAAGATATCTTAGA
AGATATAGTGTTGACTCTTACCCTCTTTGAAGATCGGGAAATGATTGAGG
AAAGACTAAAAACATACGCTCACCTGTTCGACGATAAGGTTATGAAACAG
TTAAAGAGGCGTCGCTATACGGGCTGGGGACGATTGTCGCGGAAACTTAT
CAACGGGATAAGAGACAAGCAAAGTGGTAAAACTATTCTCGATTTTCTAA
AGAGCGACGGCTTCGCCAATAGGAACTTTATGCAGCTGATCCATGATGAC
TCTTTAACCTTCAAAGAGGATATACAAAAGGCACAGGTTTCCGGACAAGG
GGACTCATTGCACGAACATATTGCGAATCTTGCTGGTTCGCCAGCCATCA
AAAAGGGCATACTCCAGACAGTCAAAGTAGTGGATGAGCTAGTTAAGGTC
ATGGGACGTCACAAACCGGAAAACATTGTAATCGAGATGGCACGCGAAAA
TCAAACGACTCAGAAGGGGCAAAAAAACAGTCGAGAGCGGATGAAGAGAA
TAGAAGAGGGTATTAAAGAACTGGGCAGCCAGATCTTAAAGGAGCATCCT
GTGGAAAATACCCAATTGCAGAACGAGAAACTTTACCTCTATTACCTACA
AAATGGAAGGGACATGTATGTTGATCAGGAACTGGACATAAACCGTTTAT
CTGATTACGACGTCGATCACATTGTACCCCAATCCTTTTTGAAGGACGAT
TCAATCGACAATAAAGTGCTTACACGCTCGGATAAGAACCGAGGGAAAAG
TGACAATGTTCCAAGCGAGGAAGTCGTAAAGAAAATGAAGAACTATTGGC
GGCAGCTCCTAAATGCGAAACTGATAACGCAAAGAAAGTTCGATAACTTA
ACTAAAGCTGAGAGGGGTGGCTTGTCTGAACTTGACAAGGCCGGATTTAT
TAAACGTCAGCTCGTGGAAACCCGCCAAATCACAAAGCATGTTGCACAGA
TACTAGATTCCCGAATGAATACGAAATACGACGAGAACGATAAGCTGATT
CGGGAAGTCAAAGTAATCACTTTAAAGTCAAAATTGGTGTCGGACTTCAG
AAAGGATTTTCAATTCTATAAAGTTAGGGAGATAAATAACTACCACCATG
CGCACGACGCTTATCTTAATGCCGTCGTAGGGACCGCACTCATTAAGAAA
TACCCGAAGCTAGAAAGTGAGTTTGTGTATGGTGATTACAAAGTTTATGA
CGTCCGTAAGATGATCGCGAAAAGCGAACAGGAGATAGGCAAGGCTACAG
CCAAATACTTCTTTTATTCTAACATTATGAATTTCTTTAAGACGGAAATC
ACTCTGGCAAACGGAGAGATACGCAAACGACCTTTAATTGAAACCAATGG
GGAGACAGGTGAAATCGTATGGGATAAGGGCCGGGACTTCGCGACGGTGA
GAAAAGTTTTGTCCATGCCCCAAGTCAACATAGTAAAGAAAACTGAGGTG
CAGACCGGAGGGTTTTCAAAGGAATCGATTCTTCCAAAAAGGAATAGTGA
TAAGCTCATCGCTCGTAAAAAGGACTGGGACCCGAAAAAGTACGGTGGCT
TCGATAGCCCTACAGTTGCCTATTCTGTCCTAGTAGTGGCAAAAGTTGAG
AAGGGAAAATCCAAGAAACTGAAGTCAGTCAAAGAATTATTGGGGATAAC
GATTATGGAGCGCTCGTCTTTTGAAAAGAACCCCATCGACTTCCTTGAGG
CGAAAGGTTACAAGGAAGTAAAAAAGGATCTCATAATTAAACTACCAAAG
TATAGTCTGTTTGAGTTAGAAAATGGCCGAAAACGGATGTTGGCTAGCGC
CGGAGAGCTTCAAAAGGGGAACGAACTCGCACTACCGTCTAAATACGTGA
ATTTCCTGTATTTAGCGTCCCATTACGAGAAGTTGAAAGGTTCACCTGAA
GATAACGAACAGAAGCAACTTTTTGTTGAGCAGCACAAACATTATCTCGA
CGAAATCATAGAGCAAATTTCGGAATTCAGTAAGAGAGTCATCCTAGCTG
ATGCCAATCTGGACAAAGTATTAAGCGCATACAACAAGCACAGGGATAAA
CCCATACGTGAGCAGGCGGAAAATATTATCCATTTGTTTACTCTTACCAA
CCTCGGCGCTCCAGCCGCATTCAAGTATTTTGACACAACGATAGATCGCA
AACGATACACTTCTACCAAGGAGGTGCTAGACGCGACACTGATTCACCAA
TCCATCACGGGATTATATGAAACTCGGATAGATTTGTCACAGCTTGGGGG
TGACGGATCCCCCAAGAAGAAGAGGAAAGTCTCGAGCGACTACAAAGACC
ATGACGGTGATTATAAAGATCATGACATCGATTACAAGGATGACGATGAC
AAGGCTGCAGGA

(SEQ ID NO: 26)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKEDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
(single underline: HNH domain; double underline:
RuvC domain)

In some embodiments, wild type Cas9 corresponds to Cas9 from Streptococcus pyogenes (NCBI Reference Sequence: NC_002737.2. SEQ ID NO: 27 (nucleotide); and Uniport Reference Sequence: Q99ZW2. SEQ ID NO: 28 (amino acid).

(SEQ ID NO: 27)
ATGGATAAGAAATACTCAATAGGCTTAGATATCGGCACAAATAGCGTCGG
ATGGGCGGTGATCACTGATGAATATAAGGTTCCGTCTAAAAAGTTCAAGG
TTCTGGGAAATACAGACCGCCACAGTATCAAAAAAAATCTTATAGGGGCT
CTTTTATTTGACAGTGGAGAGACAGCGGAAGCGACTCGTCTCAAACGGAC
AGCTCGTAGAAGGTATACACGTCGGAAGAATCGTATTTGTTATCTACAGG
AGATTTTTTCAAATGAGATGGCGAAAGTAGATGATAGTTTCTTTCATCGA
CTTGAAGAGTCTTTTTTGGTGGAAGAAGACAAGAAGCATGAACGTCATCC
TATTTTTGGAAATATAGTAGATGAAGTTGCTTATCATGAGAAATATCCAA
CTATCTATCATCTGCGAAAAAAATTGGTAGATTCTACTGATAAAGCGGAT
TTGCGCTTAATCTATTTGGCCTTAGCGCATATGATTAAGTTTCGTGGTCA
TTTTTTGATTGAGGGAGATTTAAATCCTGATAATAGTGATGTGGACAAAC
TATTTATCCAGTTGGTACAAACCTACAATCAATTATTTGAAGAAAACCCT
ATTAACGCAAGTGGAGTAGATGCTAAAGCGATTCTTTCTGCACGATTGAG
TAAATCAAGACGATTAGAAAATCTCATTGCTCAGCTCCCCGGTGAGAAGA
AAAATGGCTTATTTGGGAATCTCATTGCTTTGTCATTGGGTTTGACCCCT
AATTTTAAATCAAATTTTGATTTGGCAGAAGATGCTAAATTACAGCTTTC
AAAAGATACTTACGATGATGATTTAGATAATTTATTGGCGCAAATTGGAG
ATCAATATGCTGATTTGTTTTTGGCAGCTAAGAATTTATCAGATGCTATT
TTACTTTCAGATATCCTAAGAGTAAATACTGAAATAACTAAGGCTCCCCT
ATCAGCTTCAATGATTAAACGCTACGATGAACATCATCAAGACTTGACTC
TTTTAAAAGCTTTAGTTCGACAACAACTTCCAGAAAAGTATAAAGAAATC
TTTTTTGATCAATCAAAAAACGGATATGCAGGTTATATTGATGGGGGAGC
TAGCCAAGAAGAATTTTATAAATTTATCAAACCAATTTTAGAAAAAATGG
ATGGTACTGAGGAATTATTGGTGAAACTAAATCGTGAAGATTTGCTGCGC
AAGCAACGGACCTTTGACAACGGCTCTATTCCCCATCAAATTCACTTGGG
TGAGCTGCATGCTATTTTGAGAAGACAAGAAGACTTTTATCCATTTTTAA
AAGACAATCGTGAGAAGATTGAAAAAATCTTGACTTTTCGAATTCCTTAT
TATGTTGGTCCATTGGCGCGTGGCAATAGTCGTTTTGCATGGATGACTCG
GAAGTCTGAAGAAACAATTACCCCATGGAATTTTGAAGAAGTTGTCGATA
AAGGTGCTTCAGCTCAATCATTTATTGAACGCATGACAAACTTTGATAAA
AATCTTCCAAATGAAAAAGTACTACCAAAACATAGTTTGCTTTATGAGTA
TTTTACGGTTTATAACGAATTGACAAAGGTCAAATATGTTACTGAAGGAA
TGCGAAAACCAGCATTTCTTTCAGGTGAACAGAAGAAAGCCATTGTTGAT
TTACTCTTCAAAACAAATCGAAAAGTAACCGTTAAGCAATTAAAAGAAGA
TTATTTCAAAAAAATAGAATGTTTTGATAGTGTTGAAATTTCAGGAGTTG
AAGATAGATTTAATGCTTCATTAGGTACCTACCATGATTTGCTAAAAATT
ATTAAAGATAAAGATTTTTTGGATAATGAAGAAAATGAAGATATCTTAGA
GGATATTGTTTTAACATTGACCTTATTTGAAGATAGGGAGATGATTGAGG
AAAGACTTAAAACATATGCTCACCTCTTTGATGATAAGGTGATGAAACAG
CTTAAACGTCGCCGTTATACTGGTTGGGGACGTTTGTCTCGAAAATTGAT
TAATGGTATTAGGGATAAGCAATCTGGCAAAACAATATTAGATTTTTTGA
AATCAGATGGTTTTGCCAATCGCAATTTTATGCAGCTGATCCATGATGAT
AGTTTGACATTTAAAGAAGACATTCAAAAAGCACAAGTGTCTGGACAAGG
CGATAGTTTACATGAACATATTGCAAATTTAGCTGGTAGCCCTGCTATTA
AAAAAGGTATTTTACAGACTGTAAAAGTTGTTGATGAATTGGTCAAAGTA
ATGGGGCGGCATAAGCCAGAAAATATCGTTATTGAAATGGCACGTGAAAA
TCAGACAACTCAAAAGGGCCAGAAAAATTCGCGAGAGCGTATGAAACGAA
TCGAAGAAGGTATCAAAGAATTAGGAAGTCAGATTCTTAAAGAGCATCCT
GTTGAAAATACTCAATTGCAAAATGAAAAGCTCTATCTCTATTATCTCCA
AAATGGAAGAGACATGTATGTGGACCAAGAATTAGATATTAATCGTTTAA
GTGATTATGATGTCGATCACATTGTTCCACAAAGTTTCCTTAAAGACGAT
TCAATAGACAATAAGGTCTTAACGCGTTCTGATAAAAATCGTGGTAAATC
GGATAACGTTCCAAGTGAAGAAGTAGTCAAAAAGATGAAAAACTATTGGA
GACAACTTCTAAACGCCAAGTTAATCACTCAACGTAAGTTTGATAATTTA
ACGAAAGCTGAACGTGGAGGTTTGAGTGAACTTGATAAAGCTGGTTTTAT
CAAACGCCAATTGGTTGAAACTCGCCAAATCACTAAGCATGTGGCACAAA
TTTTGGATAGTCGCATGAATACTAAATACGATGAAAATGATAAACTTATT
CGAGAGGTTAAAGTGATTACCTTAAAATCTAAATTAGTTTCTGACTTCCG
AAAAGATTTCCAATTCTATAAAGTACGTGAGATTAACAATTACCATCATG
CCCATGATGCGTATCTAAATGCCGTCGTTGGAACTGCTTTGATTAAGAAA
TATCCAAAACTTGAATCGGAGTTTGTCTATGGTGATTATAAAGTTTATGA
TGTTCGTAAAATGATTGCTAAGTCTGAGCAAGAAATAGGCAAAGCAACCG
CAAAATATTTCTTTTACTCTAATATCATGAACTTCTTCAAAACAGAAATT
ACACTTGCAAATGGAGAGATTCGCAAACGCCCTCTAATCGAAACTAATGG
GGAAACTGGAGAAATTGTCTGGGATAAAGGGCGAGATTTTGCCACAGTGC
GCAAAGTATTGTCCATGCCCCAAGTCAATATTGTCAAGAAAACAGAAGTA
CAGACAGGCGGATTCTCCAAGGAGTCAATTTTACCAAAAAGAAATTCGGA
CAAGCTTATTGCTCGTAAAAAAGACTGGGATCCAAAAAAATATGGTGGTT
TTGATAGTCCAACGGTAGCTTATTCAGTCCTAGTGGTTGCTAAGGTGGAA
AAAGGGAAATCGAAGAAGTTAAAATCCGTTAAAGAGTTACTAGGGATCAC
AATTATGGAAAGAAGTTCCTTTGAAAAAAATCCGATTGACTTTTTAGAAG
CTAAAGGATATAAGGAAGTTAAAAAAGACTTAATCATTAAACTACCTAAA
TATAGTCTTTTTGAGTTAGAAAACGGTCGTAAACGGATGCTGGCTAGTGC
CGGAGAATTACAAAAAGGAAATGAGCTGGCTCTGCCAAGCAAATATGTGA
ATTTTTTATATTTAGCTAGTCATTATGAAAAGTTGAAGGGTAGTCCAGAA
GATAACGAACAAAAACAATTGTTTGTGGAGCAGCATAAGCATTATTTAGA
TGAGATTATTGAGCAAATCAGTGAATTTTCTAAGCGTGTTATTTTAGCAG
ATGCCAATTTAGATAAAGTTCTTAGTGCATATAACAAACATAGAGACAAA
CCAATACGTGAACAAGCAGAAAATATTATTCATTTATTTACGTTGACGAA
TCTTGGAGCTCCCGCTGCTTTTAAATATTTTGATACAACAATTGATCGTA
AACGATATACGTCTACAAAAGAAGTTTTAGATGCCACTCTTATCCATCAA
TCCATCACTGGTCTTTATGAAACACGCATTGATTTGAGTCAGCTAGGAGG
TGACTGA

(SEQ ID NO: 28)
MDKKYSIGLDIGTNSVGWAVITDEYKVPSKKFKVLGNTDRHSIKKNLIGA
LLEDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
(single underline: HNH domain; double underline:
RuvC domain)

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria. meningitidis (NCBI Ref: YP_002342100.1) or to a Cas9 from any other suitable organism.

In some embodiments, dCas9 corresponds to, or comprises in part or in whole, a Cas9 amino acid sequence having one or more mutations that inactivate the Cas9 nuclease activity. For example, in some embodiments, a dCas9 domain comprises D10A and/or H840A mutation.

dCas9 (D10A and H840A):

MDKKYSIGLAIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGDLN
PDNSDVDKLFIQLVQTYNQLFEENPINASGVDAKAILSARLSKSRRLENLIAQLPGEKKN
GLFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPHQIH
LGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEG
MRKPAFLSGEQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQKA

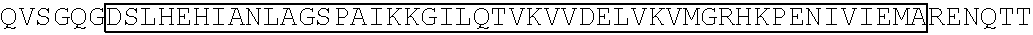

QKGQKNSRERMKRIEEGIKELGSQILKEHPVENTQLQNEKLYLYYLONGRDMYVD
QELDINRLSDYDVDAIVPQSFLKDDSIDNKVLTRSDKNRGKSDNVPSEEVVKKMKN

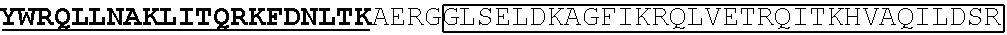

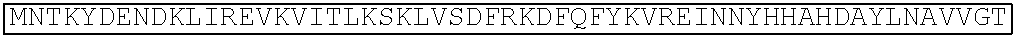

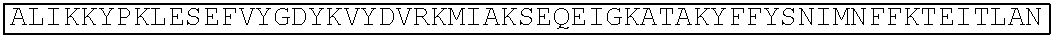

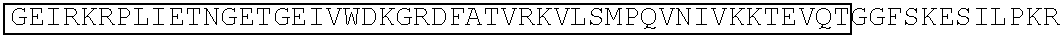

NSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSIT
GLYETRIDLSQLGGD (SEQ ID NO: 29) (single underline: HNH domain;
double underline: RuvC domain)

In some embodiments, the Cas9 domain comprises a D10A mutation, while the residue at position 840 remains a histidine in the amino acid sequence provided in SEQ ID NO: 29, or at a corresponding position in another Cas9 domain. Without wishing to be bound by any particular theory, the presence of the catalytic residue H840 restores the activity of the Cas9 to cleave the non-edited (e.g., non-deaminated) strand containing a G opposite the targeted C. Restoration of H840 (e.g., from A840) does not result in the cleavage of the target strand containing the C. Such Cas9 variants are able to generate a single-strand DNA break (nick) at a specific location based on the gRNA-defined target sequence, leading to repair of the non-edited strand, ultimately resulting in a G to A change on the non-edited strand.

In other embodiments, dCas9 variants having mutations other than D10A and H840A are provided, which, e.g., result in nuclease inactivated Cas9 (dCas9). Such mutations, by way of example, include other amino acid substitutions at D10 and H820, or other substitutions within the nuclease domains of Cas9 (e.g., substitutions in the HNH nuclease subdomain and/or the RuvC1 subdomain). In some embodiments, variants or homologues of dCas9 (e.g., variants of SEQ ID NO: 29) are provided which are at least about 70% identical, at least about 80% identical, at least about 90% identical, at least about 95% identical, at least about 98% identical, at least about 99% identical, at least about 99.5% identical, or at least about 99.9% identical to SEQ ID NO: 29. In some embodiments, variants of dCas9 (e.g., variants of SEQ ID NO: 29) are provided having amino acid sequences which are shorter, or longer than SEQ ID NO: 29, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In some embodiments, Cas9 fusion proteins as provided herein comprise the full-length amino acid sequence of a Cas9 protein, e.g., one of the Cas9 sequences provided herein. In other embodiments, however, fusion proteins as provided herein do not comprise a full-length Cas9 sequence, but only a fragment thereof. For example, in some embodiments, a Cas9 fusion protein provided herein comprises a Cas9 fragment, wherein the fragment binds crRNA and tracrRNA or sgRNA, but does not comprise a functional nuclease domain, e.g., in that it comprises only a truncated version of a nuclease domain or no nuclease domain at all. Exemplary amino acid sequences of suitable Cas9 domains and Cas9 fragments are provided herein, and additional suitable sequences of Cas9 domains and fragments will be apparent to those of skill in the art.

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1).

The term “nucleobase editors (NBEs)” or “base editors (BEs),” as used herein, refers to the Cas9 fusion proteins described herein. In some embodiments, the fusion protein comprises a nuclease-inactive Cas9 (dCas9) fused to a deaminase. In some embodiments, the fusion protein comprises a Cas9 nickase fused to a deaminase. In some embodiments, the fusion protein comprises a nuclease-inactive Cas9 fused to a deaminase and further fused to a UGI domain. In some embodiments, the fusion protein comprises a Cas9 nickase fused to a deaminase and further fused to a UGI domain. In some embodiments, the dCas9 of the fusion protein comprises a D10A and a H840A mutation, which inactivates nuclease activity of the Cas9 protein, or which renders Cas9 capable of cleaving only one strand of a nucleic acid duplex. The terms “nucleobase editors (NBEs)” and “base editors (BEs)” may be used interchangeably.

The term “uracil glycosylase inhibitor” or “UGI,” as used herein, refers to a protein that is capable of inhibiting a uracil-DNA glycosylase base-excision repair enzyme.

The term “Cas9 nickase,” as used herein, refers to a Cas9 protein that is capable of cleaving only one strand of a duplexed nucleic acid molecule (e.g., a duplexed DNA molecule). In some embodiments, a Cas9 nickase comprises a D10A mutation and has a histidine at position H840. Such a Cas9 nickase has an active HNH nuclease domain and is able to cleave the non-targeted strand of DNA, i.e., the strand bound by the gRNA. Further, such a Cas9 nickase has an inactive RuvC nuclease domain and is not able to cleave the targeted strand of the DNA, i.e., the strand where base editing is desired.

Exemplary Cas9 nickase (Cloning vector pPlatTET-gRNA2; Accession No. BAV54124).

(SEQ ID NO: 21)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITORKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

The term “linker,” as used herein, refers to a chemical group or a molecule linking two molecules or moieties, e.g., two domains of a fusion protein, such as, for example, a nuclease-inactive Cas9 domain and a nucleic acid editing domain (e.g., a deaminase domain). In some embodiments, a linker joins a gRNA binding domain of an RNA-programmable nuclease, including a Cas9 nuclease domain, and the catalytic domain of a nucleic-acid editing protein. In some embodiments, a linker joins a dCas9 and a nucleic-acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The term “nucleic acid editing domain,” as used herein refers to a protein or enzyme capable of making one or more modifications (e.g., deamination of a cytidine residue) to a nucleic acid (e.g., DNA or RNA). Exemplary nucleic acid editing domains include, but are not limited to a deaminase, a nuclease, a nickase, a recombinase, a methyltransferase, a methylase, an acetylase, an acetyltransferase, a transcriptional activator, or a transcriptional repressor domain. In some embodiments the nucleic acid editing domain is a deaminase (e.g., a cytidine deaminase, such as an APOBEC or an AID deaminase).

The term “RNA-programmable nuclease,” and “RNA-guided nuclease” are used interchangeably herein and refer to a nuclease that forms a complex with (e.g., binds or associates with) one or more RNA that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. For example, in some embodiments, domain (2) is identical or homologous to a tracrRNA as provided in Jinek et al., Science 337:816-821(2012), the entire contents of which is incorporated herein by reference. Other examples of gRNAs (e.g., those including domain 2) can be found in U.S. Provisional Patent Application, U.S. Ser. No. 61/874,682, filed Sep. 6, 2013, entitled “Switchable Cas9 Nucleases And Uses Thereof,” and U.S. Provisional Patent Application, U.S. Ser. No. 61/874,746, filed Sep. 6, 2013, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J.J., McShan W.M., Ajdic D.J., Savic D.J., Savic G., Lyon K., Primeaux C., Sezate S., Suvorov A.N., Kenton S., Lai H.S., Lin S.P., Qian Y., Jia H.G., Najar F.Z., Ren Q., Zhu H., Song L., White J., Yuan X., Clifton S.W., Roe B.A., Mclaughlin R.E., Proc. Natl. Acad. Sci. U.S.A. 98:4658-4663(2001); “CRISPR RNA maturation by trans-encoded small RNA and host factor RNase III.” Deltcheva E., Chylinski K., Sharma C.M., Gonzales K., Chao Y., Pirzada Z.A., Eckert M.R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J.A., Charpentier E. Science 337:816-821(2012), the entire contents of each of which are incorporated herein by reference.

Because RNA-programmable nucleases (e.g., Cas9) use RNA:DNA hybridization to target DNA cleavage sites, these proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W.Y. et al. Efficient genome editing in zebrafish using a CRISPR-Cas system. Nature biotechnology 31, 227-229 (2013); Jinek, M. et al. RNA-programmed genome editing in human cells. eLife 2, c00471 (2013); Dicarlo, J.E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013); the entire contents of each of which are incorporated herein by reference).

The term “host cell,” as used herein, refers to a cell that can host, replicate, and transfer a phage vector useful for a continuous evolution process as provided herein. In embodiments where the vector is a viral vector, a suitable host cell is a cell that can be infected by the viral vector, can replicate it, and can package it into viral particles that can infect fresh host cells. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the disclosure is not limited in this respect.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

The term “target site” refers to a sequence within a nucleic acid molecule that is deaminated by a deaminase or a fusion protein comprising a deaminase (e.g., a dCas9-deaminase fusion protein provided herein).

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “effective amount,” as used herein, refers to an amount of a biologically active agent that is sufficient to elicit a desired biological response. For example, in some embodiments, an effective amount of a nuclease may refer to the amount of the nuclease that is sufficient to induce cleavage of a target site specifically bound and cleaved by the nuclease. In some embodiments, an effective amount of a fusion protein provided herein, e.g., of a fusion protein comprising a nuclease-inactive Cas9 domain and a nucleic acid editing domain (e.g., a deaminase domain) may refer to the amount of the fusion protein that is sufficient to induce editing of a target site specifically bound and edited by the fusion protein. As will be appreciated by the skilled artisan, the effective amount of an agent, e.g., a fusion protein, a nuclease, a deaminase, a recombinase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

### DETAILED DESCRIPTION OF CERTAIN EMBODIMENTS

Some aspects of this disclosure provide cytidine deaminase variants (e.g., APOBEC1 protein variants) and methods, compositions, and systems for producing the same. In some embodiments, the disclosure relates to the use of soluble expression phage-assisted continuous evolution (SE-PACE) to produce cytidine deaminase protein variants. In some embodiments, cytidine deaminase protein variants described by the disclosure exhibit improved soluble expression (e.g., improved soluble expression in E. coli cells) relative to the wild-type cytidine deaminase protein from which they are derived. Some aspects of this disclosure provide fusion proteins, such as base editors (BEs) comprising a cytidine deaminase protein variant fused to a nuclease-inactive Cas9 protein, and further fused to other domains or sequences (e.g., a UGI domain).

### Cytidine Deaminase Protein Variants

Some aspects of the disclosure relate to cytidine deaminase protein variants. The disclosure is based, in part, on cytidine deaminase protein variants that are characterized by increased soluble expression (e.g., increased soluble expression in E. coli cells) relative to wild-type cytidine deaminase proteins (e.g., the cytidine deaminase protein from which the variant was evolved). In some embodiments, cytidine deaminase protein variants described by the disclosure are characterized by maintained (e.g., the same) or improved (e.g., increased) deaminase activity relative to wild-type cytidine deaminase proteins (e.g., the cytidine deaminase protein from which the variant was evolved).

Cytidine deaminase protein variants described by the disclosure are typically derived from a wild-type cytidine deaminase protein and have at least one variation in the amino acid sequence of the variant protein as compared to the amino acid sequence of the cognate wild-type cytidine deaminase protein. In some embodiments, a cytidine deaminase protein variant has at least one variation in its encoding nucleic acid sequence that results in a change in the amino acid sequence present within a cognate wild type cytidine deaminase protein. The variation in amino acid sequence generally results from a mutation, insertion, or deletion in a DNA coding sequence. Mutation of a DNA sequence can result in a nonsense mutation (e.g., a transcription termination codon (TAA, TAG, or TGA) that produces a truncated protein), a missense mutation (e.g., an insertion or deletion mutation that shifts the reading frame of the coding sequence), or a silent mutation (e.g., a change in the coding sequence that results in a codon that codes for the same amino acid normally present in the cognate protein, also referred to sometimes as a synonymous mutation). In some embodiments, mutation of a DNA sequence results in a non-synonymous (i.e., conservative, semi-conservative, or radical) amino acid substitution.

The cytidine deaminase protein can be any cytidine deaminase protein known in the art. In some embodiments, a cytidine deaminase protein variant is an apolipoprotein B mRNA-editing complex (APOBEC) family deaminase variant. In some embodiments, the deaminase is an APOBEC1 deaminase protein variant. In some embodiments, the deaminase is an APOBEC2 deaminase protein variant. In some embodiments, the deaminase is an APOBEC3 deaminase protein variant. In some embodiments, the deaminase is an APOBEC3A deaminase protein variant. In some embodiments, the deaminase is an APOBEC3B deaminase protein variant. In some embodiments, the deaminase is an APOBEC3C deaminase protein variant. In some embodiments, the deaminase is an APOBEC3D deaminase protein variant. In some embodiments, the deaminase is an APOBEC3E deaminase. In some embodiments, the deaminase is an APOBEC3F deaminase protein variant. In some embodiments, the deaminase is an APOBEC3G deaminase protein variant. In some embodiments, the deaminase is an APOBEC3H deaminase protein variant. In some embodiments, the deaminase is an APOBEC4 deaminase protein variant.

In some embodiments, a cytidine deaminase protein variant is a vertebrate cytidine deaminase protein variant. In some embodiments, the deaminase is a human, chimpanzee, gorilla, monkey, cow, dog, rat, or mouse deaminase. In some embodiments, the deaminase is a human deaminase. In some embodiments, the deaminase is a rat deaminase, e.g., rAPOBEC1.

In some embodiments, a deaminase variant is an invertebrate deaminase variant, for example, a variant of a Petromyzon marinus cytosine deaminase 1 (pmCDA1).

In some embodiments, a cytidine deaminase protein variant and a wild-type cytidine deaminase protein (e.g., rAPOBEC1) are from about 50% to about 99.9% identical, about 55% to about 95% identical, about 60% to about 90% identical, about 65% to about 85% identical, or about 70% to about 80% identical at the amino acid sequence level. In some embodiments, a cytidine deaminase protein variant comprises an amino acid sequence that is at least 50%, at least 60%, at least 70%, at least 80%, at least 90%, at least 95%, at least 99%, or at least 99.9% identical to the amino acid sequence of a wild-type cytidine deaminase protein (e.g., an APOBEC1 deaminase, such as rAPOBEC1). In some embodiments, amino acid sequence identity is based on an alignment against a reference sequence (e.g., a wild-type cytidine deaminase protein, for example, an APOBEC1 deaminase, such as rAPOBEC1) by NCBI Constraint-based Multiple Alignment Tool (COBALT), using the following parameters;

Alignment Parameters: Gap penalties-11,-1 and End-Gap penalties-5,-1, CDD Parameters: Use RPS BLAST on; Blast E-value 0.003; Find Conserved columns and Recompute on, and Query Clustering Parameters: Use query clusters on; Word Size 4; Max cluster distance 0.8; Alphabet Regular.

In some embodiments, a cytidine deaminase protein variant is about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 99.9% identical to a wild-type cytidine deaminase protein (e.g., an APOBEC1 deaminase, such as rAPOBEC1).

The amount or level of variation between a wild-type cytidine deaminase protein and a cytidine deaminase protein variant can also be expressed as the number of mutations present in the amino acid sequence encoding the cytidine deaminase protein variant relative to the amino acid sequence encoding the wild-type cytidine deaminase protein. In some embodiments, an amino acid sequence encoding a cytidine deaminase protein variant comprises between about 1 mutation and about 100 mutations, about 10 mutations and about 90 mutations, about 20 mutations and about 80 mutations, about 30 mutations and about 70 mutations, or about 40 and about 60 mutations relative to an amino acid sequence encoding a wild-type cytidine deaminase protein (e.g., an APOBEC1 protein, such as rAPOBEC1). In some embodiments, an amino acid sequence encoding a cytidine deaminase protein variant comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mutations relative to an amino acid sequence encoding a wild-type cytidine deaminase protein (e.g., an APOBEC1 protein, such as rAPOBEC1). In some embodiments, an amino acid sequence of a cytidine deaminase protein variant comprises more than 100 mutations relative to an amino acid sequence of a wild-type cytidine deaminase protein.

Particular combinations of mutations present in an amino acid sequence encoding a cytidine deaminase protein variant can be referred to as the “genotype” of the cytidine deaminase protein variant. For example, a cytidine deaminase protein variant genotype may comprise the mutations F113C, A123E, and F205S, relative to a wild-type cytidine deaminase protein rAPOBEC1 (e.g., SEQ ID NO: 15). In some embodiments, a cytidine deaminase protein variant genotype may comprise the mutations G45D, Y75H, F113C, A123E, H166N, and F205S, relative to a wild-type cytidine deaminase protein rAPOBEC1 (e.g., SEQ ID NO: 15). In some embodiments, a cytidine deaminase protein variant genotype may comprise the mutations F113C, A123E, A165T, and F205S, relative to a wild-type cytidine deaminase protein rAPOBEC1 (e.g., SEQ ID NO: 15). Aspects of the disclosure relate to the discovery that cytidine deaminase protein variants having a mutation at position E63 (relative to wild-type rAPOBEC1; SEQ ID NO: 15) are characterized by a loss of deaminase function. Accordingly, in some embodiments, a cytidine deaminase protein variant does not comprise (e.g., lacks) a mutation at the position corresponding to position E63 of wild-type rAPOBEC1 (SEQ ID NO: 15). In some embodiments, a cytidine deaminase protein variant does not comprise an E63A mutation (at a position corresponding to position E63 of rAPOBEC1, SEQ ID NO: 15).

rAPOBEC1
(SEQ ID NO: 15)
MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLK

hAPOBEC1
(SEQ ID NO: 30)
MTSEKGPSTGDPTLRRRIEPWEFDVFYDPRELRKEACLLYEIKWGMSRKI
WRSSGKNTTNHVEVNFIKKFTSERDFHPSMSCSITWFLSWSPCWECSQAI
REFLSRHPGVTLVIYVARLFWHMDQQNRQGLRDLVNSGVTIQIMRASEYY
HCWRNFVNYPPGDEAHWPQYPPLWMMLYALELHCIILSLPPCLKISRRWQ
NHLTFFRLHLQNCHYQTIPPHILLATGLIHPSVAWR

### Base Editing Proteins

Some aspects of the disclosure relate to fusion proteins (e.g., base editing proteins) comprising (i) a RNA-programmable nuclease (e.g., a Cas9 domain); and (ii) a nucleic acid editing domain (e.g., a cytidine deaminase protein variant as described by the disclosure). In some embodiments, a Cas9 domain is a nuclease-inactive Cas9 domain (dCas9), and comprises an amino acid sequence that is at least 80%, at least 85%, at least 90%, at least 92%, at least 95%, at least 96%, at least 97%, at least 98%, at least 99%, or at least 99.5% identical to the amino acid sequence of a Cas9 protein (e.g., SEQ ID NO: 20). Mutations that render the nuclease activity of Cas9 inactive are known in the art. For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28;152(5):1173-83 (2013)). In some embodiments, the dCas9 comprises a D10A mutation. In some embodiments, the dCas9 comprises a H840A mutation. In some embodiments, a dCas9 comprises both D10A and H840A mutations. It is to be understood that other mutations that inactivate the nuclease activity of Cas9 may also be included in the dCas9 of this disclosure. The above-noted mutations and additional dCas9 mutations are described, for example, in International PCT Application No. PCT/US2016/058344, published on Apr. 27, 2017 as WO 2017/070632; International PCT Application No. PCT/US2016/058345, published on Apr. 27, 2017 as WO 2017/070633; and International PCT Application No. PCT/US2017/045381, published on Feb. 8, 2018 as WO 2018/027078, the entire contents of each application which are incorporated herein by reference.

The Cas9 or dCas9 domains disclosed herein, may be a full-length Cas9, or a fragment thereof. In some embodiments, proteins comprising Cas9, or fragments thereof, are referred to as “Cas9 variants.” A Cas9 variant shares homology to Cas9, or a fragment thereof. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9, e.g., a Cas9 comprising the amino acid sequence of SEQ ID NO: 24.

Any of the Cas9 fusion proteins of this disclosure may further comprise a nucleic acid editing domain (e.g., an enzyme that is capable of modifying nucleic acid, such as a deaminase). In some embodiments, the nucleic acid editing domain is a DNA-editing domain.

In some embodiments, the Cas9 domain and the nucleic acid editing-editing domain are fused via a linker. In some embodiments, the linker comprises a (GGGS)ₙ (SEQ ID NO: 31), a (GGGGS)ₙ (SEQ ID NO: 32), a (G)ₙ, an (EAAAK)ₙ (SEQ ID NO: 33), a (GGS)ₙ, an SGSETPGTSESATPES (SEQ ID NO: 34) motif (sec, e.g., Guilinger JP, Thompson DB, Liu DR. Fusion of catalytically inactive Cas9 to FokI nuclease improves the specificity of genome modification. Nat. Biotechnol. 2014; 32(6): 577-82; the entire contents are incorporated herein by reference), or an (XP)ₙ motif, or a combination of any of these, wherein n is independently an integer between 1 and 30. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if more than one linker or more than one linker motif is present, any combination thereof. In some embodiments, the linker comprises a (GGS), motif, wherein n is 1,2, 3, 4, 5, 6, 7, 8, 9, 10, 11, 12, 13, 14 or 15. In some embodiments, the linker comprises a (GGS), motif, wherein n is 1, 3, or 7. In some embodiments, the linker comprises the amino acid sequence SGSETPGTSESATPES (SEQ ID NO: 34). Additional suitable linker motifs and linker configurations will be apparent to those of skill in the art. In some embodiments, suitable linker motifs and configurations include those described in Chen et al., Fusion protein linkers: property, design and functionality. Adv Drug Deliv Rev. 2013; 65(10): 1357-69, the entire contents of which are incorporated herein by reference. Additional suitable linker sequences will be apparent to those of skill in the art based on the instant disclosure. In some embodiments, the general architecture of exemplary Cas9 fusion proteins provided herein comprises the structure:

[NH₂]-[nucleic acid editing domain]-[Cas9]-[COOH] or

[NH₂]-[nucleic acid editing domain]-[linker]-[Cas9]-[COOH],

wherein NH₂ is the N-terminus of the fusion protein, and COOH is the C-terminus of the fusion protein.

The fusion proteins of the present disclosure may comprise one or more additional features. For example, in some embodiments, the fusion protein comprises a nuclear localization sequence (NLS). In some embodiments, the NLS of the fusion protein is localized between the nucleic acid editing domain and the Cas9 domain. In some embodiments, the NLS of the fusion protein is localized C-terminal to the Cas9 domain.

Other exemplary features that may be present are localization sequences, such as cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, the nucleic acid editing domain is a cytidine deaminase protein variant. For example, in some embodiments, the general architecture of exemplary Cas9 fusion proteins with a deaminase domain comprises the structure:

[NH₂]-[NLS]-[deaminase]-[Cas9]-[COOH],

[NH₂]-[Cas9]-[deaminase]-[COOH],

[NH₂]-[deaminase]-[Cas9]-[COOH], or

[NH₂]-[deaminase]-[Cas9]-[NLS]-[COOH]

wherein NLS is a nuclear localization sequence, NH₂ is the N-terminus of the fusion protein, and COOH is the C-terminus of the fusion protein. Nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, a NLS comprises the amino acid sequence PKKKRKV (SEQ ID NO: 35) or MDSLLMNRRKFLYQFKNVRWAKGRRETYLC (SEQ ID NO: 36). In some embodiments, a linker is inserted between the Cas9 and the deaminase. In some embodiments, the NLS is located C-terminal of the Cas9 domain. In some embodiments, the NLS is located N-terminal of the Cas9 domain. In some embodiments, the NLS is located between the deaminase and the Cas9 domain. In some embodiments, the NLS is located N-terminal of the deaminase domain. In some embodiments, the NLS is located C-terminal of the deaminase domain.

Some aspects of this disclosure relate to the recognition that the activity of cytosine deaminase protein variants, such as rAPOBEC1 variants described herein can be directed to a specific site in genomic DNA. Without wishing to be bound by any particular theory, advantages of using Cas9 as a recognition agent include (1) the sequence specificity of Cas9 can be easily altered by simply changing the sgRNA sequence; and (2) Cas9 binds to its target sequence by denaturing the dsDNA, resulting in a stretch of DNA that is single-stranded and therefore a viable substrate for the deaminase. It should be understood that other catalytic domains, or catalytic domains from other deaminases, can also be used to generate fusion proteins with Cas9, and that the disclosure is not limited in this regard. a person of skill in the art will be able to design suitable guide RNAs to target the fusion proteins to a target sequence that comprises a nucleotide to be deaminated.

Some aspects of the disclosure relate to fusion proteins that comprise a uracil glycosylase inhibitor (UGI) domain. In some embodiments, any of the fusion proteins provided herein that comprise a Cas9 domain (e.g., a nuclease active Cas9 domain, a nuclease inactive dCas9 domain, or a Cas9 nickase) may be further fused to a UGI domain either directly or via a linker. Some aspects of this disclosure provide deaminase-dCas9 fusion proteins, deaminase-nuclease active Cas9 fusion proteins and deaminase-Cas9 nickase fusion proteins with increased nucleobase editing efficiency. Without wishing to be bound by any particular theory, cellular DNA-repair response to the presence of U:G heteroduplex DNA may be responsible for the decrease in nucleobase editing efficiency in cells. For example, uracil DNA glycosylase (UDG) catalyzes removal of U from DNA in cells, which may initiate base excision repair, with reversion of the U:G pair to a C:G pair as the most common outcome. In some embodiments, Uracil DNA Glycosylase Inhibitor (UGI) may inhibit human UDG activity. Thus, this disclosure contemplates a fusion protein comprising dCas9-nucleic acid editing domain further fused to a UGI domain. This disclosure also contemplates a fusion protein comprising a Cas9 nickase-nucleic acid editing domain further fused to a UGI domain. It should be understood that the use of a UGI domain may increase the editing efficiency of a nucleic acid editing domain that is capable of catalyzing a C to U change. For example, fusion proteins comprising a UGI domain may be more efficient in deaminating C residues. In some embodiments, the fusion protein comprises the structure:

[deaminase]-[optional linker sequence]-[dCas9]-[optional linker sequence]-[UGI];

[deaminase]-[optional linker sequence]-[UGI]-[optional linker sequence]-[dCas9];

[UGI]-[optional linker sequence]-[deaminase]-[optional linker sequence]-[dCas9];

[UGI]-[optional linker sequence]-[dCas9]-[optional linker sequence]-[deaminase];

[dCas9]-[optional linker sequence]-[deaminase]-[optional linker sequence]-[UGI]; or

[dCas9]-[optional linker sequence]-[UGI]-[optional linker sequence]-[deaminase].

In other embodiments, the fusion protein comprises the structure:

[deaminase]-[optional linker sequence]-[Cas9 nickase]-[optional linker sequence]-[UGI];

[deaminase]-[optional linker sequence]-[UGI]-[optional linker sequence]-[Cas9 nickase];

[UGI]-[optional linker sequence]-[deaminase]-[optional linker sequence]-[Cas9 nickase];

[UGI]-[optional linker sequence]-[Cas9 nickase]-[optional linker sequence]-[deaminase];

[Cas9 nickase]-[optional linker sequence]-[deaminase]-[optional linker sequence]-[UGI]; or

[Cas9 nickase]-[optional linker sequence]-[UGI]-[optional linker sequence]-[deaminase].

In some embodiments, the fusion proteins provided herein do not comprise a linker sequence. In some embodiments, one or both of the optional linker sequences are present.

In some embodiments, the “-” used in the general architecture above indicates the presence of an optional linker sequence. In some embodiments, the fusion proteins comprising a UGI further comprise a nuclear targeting sequence, for example a nuclear localization sequence. In some embodiments, fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the UGI protein. In some embodiments, the NLS is fused to the C-terminus of the UGI protein. In some embodiments, the NLS is fused to the N-terminus of the Cas9 protein. In some embodiments, the NLS is fused to the C-terminus of the Cas9 protein. In some embodiments, the NLS is fused to the N-terminus of the deaminase. In some embodiments, the NLS is fused to the C-terminus of the deaminase. In some embodiments, the NLS is fused to the N-terminus of the second Cas9. In some embodiments, the NLS is fused to the C-terminus of the second Cas9. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. In some embodiments, the NLS comprises the amino acid PKKKRKV (SEQ ID NO: 35) or MDSLLMNRRKFLYQFKNVRWAKGRRETYLC (SEQ ID NO: 36).

In some embodiments, a UGI domain comprises a wild-type UGI or a UGI as set forth in SEQ ID NO: 22. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 22. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 22. In some embodiments, a UGI comprises an amino acid sequence homologous to the amino acid sequence set forth in SEQ ID NO: 22 or an amino acid sequence homologous to a fragment of the amino acid sequence set forth in SEQ ID NO: 22. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 22. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 22. In some embodiments, the UGI comprises the following amino acid sequence:

>sp|P14739|UNGI_BPPB2 Uracil-DNA glycosylase
inhibitor
(SEQ ID NO: 22)
MTNLSDIIEKETGKQLVIQESILMLPEEVEEVIGNKPESDILVHTAYDES
TDENVMLLTSDAPEYKPWALVIQDSNGENKIKML

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264:1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346(1999), the entire contents of each are incorporated herein by reference.

### Complexes With Guide RNAs (gRNAs)

Some aspects of this disclosure provide complexes comprising any of the fusion proteins provided herein, and a guide RNA bound to a Cas9 domain (e.g., a dCas9, a nuclease active Cas9, or a Cas9 nickase) of the fusion protein.

In some embodiments, the guide RNA is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In some embodiments, the target sequence is a DNA sequence. In some embodiments, the target sequence is a sequence in the genome of a mammal. In some embodiments, the target sequence is a sequence in the genome of a human. In some embodiments, the 3′ end of the target sequence is immediately adjacent to a canonical PAM sequence (NGG). In some embodiments, the guide RNA is complementary to a sequence associated with a disease or disorder.

### Methods of Use

Some aspects of this disclosure provide methods of using the cytidine deaminase protein variants, fusion proteins, and/or complexes provided herein. For example, some aspects of this disclosure provide methods comprising contacting a DNA molecule (a) with any of the cytidine deaminase protein variants or fusion proteins provided herein, and with at least one guide RNA, wherein the guide RNA is about 15-100 nucleotides long and comprises a sequence of at least 10 contiguous nucleotides that is complementary to a target sequence; or (b) with a RNA-programmable nuclease (e.g., a Cas9 protein, a Cas9 fusion protein, or a Cas9 protein) or fusion protein complex with at least one gRNA as provided herein. In some embodiments, the 3′-end of the target sequence is not immediately adjacent to a canonical PAM sequence (NGG). In some embodiments, the 3′-end of the target sequence is immediately adjacent to an AGC, GAG, TTT, GTG, or CAA sequence.

In some embodiments, the target DNA sequence comprises a sequence associated with a disease or disorder. In some embodiments, the target DNA sequence comprises a point mutation associated with a disease or disorder. In some embodiments, the activity of the cytidine deaminase protein variant, the Cas9 fusion protein, or the complex results in a correction of the point mutation. In some embodiments, the target DNA sequence comprises a T→C point mutation associated with a disease or disorder, and wherein the deamination of the mutant C base results in a sequence that is not associated with a disease or disorder. In some embodiments, the target DNA sequence encodes a protein and wherein the point mutation is in a codon and results in a change in the amino acid encoded by the mutant codon as compared to the wild-type codon. In some embodiments, the deamination of the mutant C results in a change of the amino acid encoded by the mutant codon. In some embodiments, the deamination of the mutant C results in the codon encoding the wild-type amino acid. In some embodiments, the contacting is in vivo in a subject. In some embodiments, the subject has or has been diagnosed with a disease or disorder. In some embodiments, the disease or disorder is cystic fibrosis, phenylketonuria, epidermolytic hyperkeratosis (EHK), Charcot-Marie-Toot disease type 4J, neuroblastoma (NB), von Willebrand disease (vWD), myotonia congenital, hereditary renal amyloidosis, dilated cardiomyopathy (DCM), hereditary lymphedema, familial Alzheimer's disease, HIV, Prion disease, chronic infantile neurologic cutaneous articular syndrome (CINCA), desmin-related myopathy (DRM), a neoplastic disease associated with a mutant PI3KCA protein, a mutant CTNNB1 protein, a mutant HRAS protein, or a mutant p53 protein.

Some embodiments provide methods for using fusion proteins (e.g., base editors) provided herein. In some embodiments, the fusion protein is used to introduce a point mutation into a nucleic acid by deaminating a target nucleobase, e.g., a C residue. In some embodiments, the deamination of the target nucleobase results in the correction of a genetic defect, e.g., in the correction of a point mutation that leads to a loss of function in a gene product. In some embodiments, the genetic defect is associated with a disease or disorder, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease or disorder. For example, in some embodiments, methods are provided herein that employ a Cas9 DNA editing fusion protein to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

In some embodiments, the purpose of the methods provide herein is to restore the function of a dysfunctional gene via genome editing. The Cas9 deaminase fusion proteins provided herein can be validated for gene editing-based human therapeutics in vitro, e.g., by correcting a disease-associated mutation in human cell culture. It will be understood by the skilled artisan that the fusion proteins provided herein, e.g., the fusion proteins comprising a Cas9 domain and a nucleic acid deaminase domain can be used to correct any single point T->C or A->G mutation. In the first case, deamination of the mutant C back to U corrects the mutation, and in the latter case, deamination of the C that is base-paired with the mutant G, followed by a round of replication, corrects the mutation.

It will be apparent to those of skill in the art that in order to target a Cas9:nucleic acid editing enzyme/domain fusion protein as disclosed herein to a target site, e.g., a site comprising a point mutation to be edited, it is typically necessary to co-express the Cas9:nucleic acid editing enzyme/domain fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein. In some embodiments, the guide RNA comprises a structure 5′-[guide sequence] guuuuagagcuagaaauagcaaguuaaaauaaggcuaguccguuaucaacuugaaaaaguggcaccgagucggugcuuuuu-3′ (SEQ ID NO: 37), wherein the guide sequence comprises a sequence that is complementary to the target sequence.

The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited.

Some aspects of the disclosure provide methods for editing a nucleic acid. In some embodiments, the method is a method for editing a nucleobase of a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a Cas9 domain fused to a cytidine deaminase protein variant) and a guide nucleic acid (e.g., gRNA), wherein the target region comprises a targeted nucleobase pair; b) inducing strand separation of said target region; c) converting a first nucleobase of said target nucleobase pair in a single strand of the target region to a second nucleobase; and d) cutting no more than one strand of said target region, where a third nucleobase complementary to the first nucleobase base is replaced by a fourth nucleobase complementary to the second nucleobase; and the method results in less than 20% indel formation in the nucleic acid.

### Vectors and Kits

Some aspects of this disclosure provide expression constructs encoding gene products that select for a desired physiochemical characteristic or desired function of an evolved cytidine deaminase protein, such as rAPOBEC1 in a host cell, e.g., in a bacterial host cell. In some embodiments, a selection system comprises one or more gene products encoded by a nucleic acid (e.g., an isolated nucleic acid). In some embodiments, one or more nucleic acids that are operably linked comprise an expression construct. Expression constructs are sometimes also referred to as vectors. In some embodiments, the expression constructs are plasmids.

In some aspects, the disclosure relates to expression vectors (e.g., plasmids) comprising a gene of interest to be evolved (e.g., a cytidine deaminase protein, such as rAPOBEC1) fused to a sequence encoding a T7 RNA polymerase (RNAP) N-terminal domain. In some embodiments, the plasmid is a selection plasmid (e.g., selection phagemid). In some embodiments, the expression construct comprises a nucleic acid encoding a cytidine deaminase protein is contiguous (e.g., operably linked) to the nucleic acid sequence encoding the T7 RNAP N-terminal domain. In some embodiments, the 3′-end of the nucleic acid encoding the cytidine deaminase protein is contiguous (e.g., operably linked) to the 5′-end of the nucleic acid encoding the T7 RNAP N-terminal domain. In some embodiments, the expression construct further comprises a promoter, such as a P_{BAD} promoter.

An N-terminal domain of a T7 RNAP may comprise between about 1% and about 99% (e.g., any percentage between 1% and 99%), about 10% and about 80%, or about 30% and about 60% of the amino acid residues of a full-length T7 RNAP (e.g., about 1% to about 99% of the amino acid residues of NCBI Accession No. NC_041960.1). In some embodiments, a T7 RNAP N-terminal domain comprises between 1 and 800 amino acid (e.g., any integer between 1 and 800, inclusive) truncations relative to the C-terminus of wild-type T7 RNAP (e.g., NCBI Accession No. NC_041960.1). For example, in some embodiments, an N-terminal domain of T7 RNAP comprises amino acid residues 1-50, 1-100, 1-150, 1-300, 1-400, 1-500, or 1-800 of NCBI Accession No. NC_041960.1.

In some aspects, the disclosure relates to expression vectors (e.g., plasmids) comprising an isolated nucleic acid having a sequence encoding a T7 RNA polymerase (RNAP) C-terminal domain. In some embodiments, the expression vector (e.g., plasmid) is an accessory plasmid. Without wishing to be bound by any particular theory, expression of a C-terminal domain of T7 RNAP in a cell (e.g., a host cell) in which a properly-folded fusion protein (e.g., comprising a gene of interest fused to a T7 RNAP N-terminal domain) is expressed, results in reconstitution of a functional T7 RNAP. A C-terminal domain of a T7 RNAP may comprise between about 1% and about 99% (e.g., any percentage between 1% and 99%), about 10% and about 80%, or about 30% and about 60% of the amino acid residues of a full-length T7 RNAP (e.g., about 1% to about 99% of the amino acid residues of NCBI Accession No. NC_041960.1). In some embodiments, a T7 RNAP C-terminal domain comprises between 1 and 800 amino acid (e.g., any integer between 1 and 800, inclusive) truncations relative to the N-terminus of wild-type T7 RNAP (e.g., NCBI Accession No. NC_041960.1). For example, in some embodiments, an N-terminal domain of T7 RNAP comprises amino acid residues 50-883, 100-883, 200-883, 400-884, or 500-883 of NCBI Accession No. NC_041960.1.

In some aspects, the disclosure relates to expression vectors (e.g., plasmids) comprising a cytidine deaminase protein to be evolved fused to a sequence encoding a T7 RNA polymerase (RNAP) N-terminal domain further comprises a nucleic acid encoding a split intein portion (e.g., fragment). An “intein” refers to a protein that is able to self-catalytically excise itself and join the remaining protein fragments (e.g., exteins) by the process of protein splicing. Generally, the self-splicing function of inteins makes them useful tools for engineering trans-spliced recombinant proteins, as described in U.S. Publication No. 2003-0167533, the entire contents of which are incorporated herein by reference. For example, expressing (i) a nucleic acid sequence encoding a N-terminal intein fragment (or portion) operably linked to a nucleic acid encoding a first protein fragment (A), and (ii) a nucleic acid encoding a C-terminal intein fragment (or portion) operably linked to a nucleic acid encoding a second protein fragment (B), in a cell would result, in some embodiments, in trans-splicing of the inteins within the cell to produce a fusion molecule comprising (in the following order) “A-B”.

Inteins are present in both prokaryotic and eukaryotic organisms. In some embodiments, an intein is a bacterial intein, such as a cyanobacterial intein (e.g., intein from Synechocystis or Nostoc). In some embodiments, the intein is a Nostoc punctiforme (Npu) intein, for example as described in Ocemig et al. (2009) FEBS Lett. 583(9): 1451-6.

In some embodiments, an expression construct described herein comprises a nucleic acid encoding a split intein portion (e.g., a split intein N-terminal portion or split intein C-terminal portion) operably linked to a nucleic acid encoding a gene required for the production of infectious phage particles, such as gIII protein (pIII protein), or a portion (e.g., fragment) thereof. In some embodiments, the split intein portion is a split intein C-terminal portion (e.g., a Npu split intein C-terminal portion). In some embodiments, the split intein C-terminal portion is positioned upstream of (e.g., 5′ relative to) the nucleic acid encoding the gene required for the production of infectious phage particles, or portion thereof. In some embodiments, the split intein portion is a split intein N-terminal portion (e.g., a Npu split intein N-terminal portion). In some embodiments, the split intein N-terminal portion is positioned downstream of (e.g., 3′ relative to) the nucleic acid encoding the gene required for the production of infectious phage particles, or portion thereof.

In some embodiments, the nucleic acid encoding a gene required for the production of infectious phage particles, such as gIII protein (pIII protein), is truncated (e.g., missing one or more nucleic acid bases relative to a full-length gene encoding pIII protein). In some embodiments, the nucleic acid is 1, 2, 3, 4, 5, 6, 7, 8, 9, 10, or more nucleic acid bases shorter than a full-length gene encoding pIII protein. It should be appreciated that the nucleic acid encoding truncated pIII protein may be truncated at either the 5′-end or the 3′-end. However, in preferred embodiments, the nucleic acid is truncated at the 5′-end. In some embodiments, the gene product encoded by the truncated nucleic acid lacks a full-length N-terminal signal peptide. In some embodiments, the gene product lacks amino acid residues 1-10 of a gIII protein signal peptide. In some embodiments, the gene product lacks amino acid residues 1-11, 1-12, 1-13, 1-14, 1-15, 1-16, 1-17, or 1-18 (e.g., the entire signal peptide) of pIII. In some embodiments, a portion of a pIII protein comprises the sequence set forth in SEQ ID NO: 38 (MKKLLPAIP). In some embodiments, a portion of a pIII protein comprises the sequence set forth in SEQ ID NO: 39:

VVPFYSHSAETVESCLAKPHTENSFTNVWKDDKTLDRYANYEGCLWNATG
VVVCTGDETQCYGTWVPIGLAIPENEGGGSEGGGSEGGGSEGGGTKPPEY
GDTPIPGYTYINPLDGTYPPGTEQNPANPNPSLEESQPLNTFMFQNNRFR
NRQGALTVYTGTVTQGTDPVKTYYQYTPVSSKAMYDAYWNGKFRDCAFHS
GFNEDPFVCEYQGQSSDLPQPPVNAGGGSGGGSGGGSEGGGSEGGGSEGG
GSEGGGSGGGSGSGDFDYEKMANANKGAMTENADENALQSDAKGKLDSVA
TDYGAAIDGFIGDVSGLANGNGATGDFAGSNSQMAQVGDGDNSPLMNNFR
QYLPSLPQSVECRPFVFGAGKPYEFSIDCDKINLFRGVFAFLLYVATFMY
VFSTFANILRNKES.

In some embodiments, expression constructs (e.g., plasmids), or combinations of expression constructs (e.g. combinations of plasmids) described herein may be used as a bacterial 2-hybrid system to positively select for evolved cytidine deaminase protein variants having desirable physiochemical characteristics (e.g., solubility, stability, etc.) or desirable function. In the context of PACE, bacterial 2-hybrid selections have been described, for example in International PCT Application, PCT/US2016/043559, filed Jul. 22, 2016, published as WO 2017/015559 on Jan. 26, 2017, and Badran et al. (2016) Nature 533:58-63, the entire contents of each of which are incorporated herein by reference.

In some embodiments, a bacterial 2-hybrid system comprises a first expression construct comprising a nucleic acid encoding a split intein C-terminal portion fused (e.g., operably linked) to a gene encoding a truncated gIII as described herein, and a second expression construct comprising a nucleic acid encoding a split intein N-terminal portion fused (e.g., operably linked) to a gene encoding a truncated gIII, where expression of the first expression construct and the second expression construct in a cell results in reconstitution (e.g., protein splicing) of a full-length functional pIII protein. In some embodiments, the first expression construct is operably linked to a promoter, such as a T7 RNAP promoter. In some embodiments, the first expression construct or the second expression construct further comprises a nucleic acid encoding a phage repressor protein, for example 434 cI repressor protein. In some embodiments, the first expression construct or the second expression construct is encodes a fusion protein comprising 434 cI repressor protein and the SH2 domain of an ABL1 kinase.

The first expression construct and the second expression construct can be located on the same vector (e.g., plasmid) or on separate vectors (e.g., different plasmids). In some embodiments, the vector is an accessory plasmid (AP). In some embodiments, a bacterial 2-hybrid system comprises a third expression construct comprising a nucleic acid encoding a cytidine deaminase protein, such as rAPOBEC1, to be evolved, T7 RNAP N-terminal domain, and an RNA polymerase subunit. In some embodiments, the RNA polymerase is RNA polymerase subunit omega (RpoZ). In some embodiments, the first expression construct, second expression construct, or third expression construct comprises a nucleic acid encoding a T7 RNAP C-terminal domain.

In some aspects, the disclosure provides expression constructs (e.g., plasmids) comprising an isolated nucleic acid encoding a leucine zipper domain of the yeast GCN4 transcription factor (“GCN4 tag”) fused upstream of the protein of the cytidine deaminase protein to be evolved. In some embodiments the expression construct is a selection plasmid (SP). For example, using a split intein system described elsewhere in the disclosure, two simultaneous PACE selections are run: the first is a split T7 RNAP-based solubility selection, and the second is a protein binding selection with an accessory plasmid (AP) encoding the anti-GCN4 scFv m3, which recognizes the GCN4 tag. The T7 RNAP based solubility selection evolves for increased protein solubility, while the GCN4 tag protein binding selection prevents potential cheating in the same way as an activity-dependent selection.

Some aspects of this disclosure provide a cell comprising an expression construct or a plasmid as provided herein. In some embodiments, the cell is a bacterial cell. In some embodiments, the cell further comprises a selection plasmid or an accessory plasmid. In some embodiments, the cell is a host cell for a bacteriophage. In some embodiments, the cell is an E. coli cell. In some embodiments, the cell is comprised in a lagoon.

Some aspects of this disclosure provide a vector system comprising: (i) a selection phagemid comprising an isolated nucleic acid comprising an expression construct encoding a fusion protein comprising, in the following order: a GCN4 peptide, a cytidine deaminase protein, a T7 RNA polymerase, and an E. coli RNA polymerase omega subunit (rpoZ); (ii) a first accessory plasmid comprising an isolated nucleic acid comprising an expression construct encoding a RNA polymerase I promoter operably linked to an expression cassette comprising, in the following order: a sequence encoding a M13 phage gIII protein signal peptide, and a sequence encoding a Npu split intein N-terminal portion, wherein the sequence encoding the gIII protein signal peptide lacks one or more nucleic acid bases of the signal peptide domain; and (iii) a second accessory plasmid comprising an isolated nucleic acid comprising an expression construct encoding a T7 promoter operably linked to an expression cassette comprising, in the following order: a sequence encoding a Npu split intein C-terminal portion, and a sequence encoding a M13 phage gIII protein, wherein the sequence encoding the gIII protein lacks one or more nucleic acid bases in the signal peptide domain.

In some embodiments, a vector system is provided as part of a kit, which is useful, in some embodiments, for performing SE-PACE to produce cytidine deaminase protein variants. For example, in some embodiments, a kit comprises a first container housing the selection phagemid of the vector system, a second container housing the first accessory plasmid of the vector system, and a third container housing the second accessory plasmid of the vector system. In some embodiments, a kit further comprises a mutagenesis plasmid. Mutagenesis plasmids for PACE are generally known in the art, and are described, for example in International PCT Application No. PCT/US2016/027795, filed Sep. 16, 2016, published as WO 2016/168631, the entire contents of which are incorporated herein by reference. In some embodiments, the kit further comprises a set of written or electronic instructions for performing SE-PACE.

### EXAMPLE

General methods: Antibiotics (Gold Biotechnology) were used at the following working concentrations: carbenicillin 50 μg/mL, spectinomycin 50 μg/mL, chloramphenicol 25 μg/mL, kanamycin 50 μg/mL, tetracycline 10 μg/mL, streptomycin 50 μg/mL. HyClone water (GE Healthcare Life Sciences) was used for PCR reactions and cloning. For all other experiments, water was purified using a MilliQ purification system (Millipore). Phusion U Hot Start DNA polymerase (Thermo Fisher Scientific) was used for all PCRs. Plasmids and selection phages (SPs) were cloned by USER assembly. Genes were obtained as synthesized gBlock gene fragments from Integrated DNA Technologies or PCR amplified directly from E. coli genomic DNA. Plasmids were cloned and amplified using either Mach1 (Thermo Fisher Scientific) or Turbo (New England BioLabs) cells. Unless otherwise noted, plasmid or SP DNA was amplified using the Illustra Templiphi 100 Amplification Kit (GE Healthcare Life Sciences) prior to Sanger sequencing.

Preparation and transformation of chemically competent cells: Strain S2060 was used in all luciferase, phage propagation, and plaque assays, and in all PACE experiments. To prepare competent cells, an overnight culture was diluted 1000-fold into 50 mL of 2×YT media (United States Biologicals) supplemented with tetracycline and streptomycin and grown at 37° C. with shaking at 230 RPM to OD₆₀₀˜0.4-0.6. Cells were pelleted by centrifugation at 4000 g for 10 minutes at 420 C. The cell pellet was then resuspended by gentle stirring in 2 mL of ice-cold LB media (United States Biologicals) 2 mL of 2×TSS (LB media supplemented with 5% v/v DMSO, 10% w/v PEG 3350, and 20 mM MgCl₂) was added. The cell suspension was stirred to mix completely, aliquoted and frozen on dry ice, and stored at −80° C. until use.

To transform cells, 100 μL of competent cells thawed on ice was added to a pre-chilled mixture of plasmid (2 μL each; up to 3 plasmids per transformation) in 95 μL KCM solution (100 mM KCl, 30 mM CaCl₂, and 50 mM MgCl₂ in H₂O) and stirred gently with a pipette tip. The mixture was incubated on ice for 10 min and heat shocked at 42° C. for 75 s before 600 μL of SOC media (New England BioLabs) was added. Cells were allowed to recover at 3720 C. with shaking at 230 RPM for 1.5 h, streaked on 2×YT media+1.5% agar (United States Biologicals) plates containing the appropriate antibiotics, and incubated at 37° C. for 16-18 h.

Phage propagation assay: S2060 cells were transformed with the accessory plasmids AP(s) of interest. Overnight cultures of single colonies grown in 2×YT media supplemented with maintenance antibiotics were diluted 1000-fold into DRM media with maintenance antibiotics and grown at 37° C. with shaking at 230 RPM to OD₆₀₀˜0.4-0.6. Cells were then infected with SP at a starting titer of 5×10⁴ pfu/mL. Cells were incubated for another 16-18 h at 37° C. with shaking at 230 RPM, then centrifuged at 8000 g for 2 min. The supernatant containing phage was removed and stored at 4° C. until use.

Plaque assay: S2060 cells were transformed with the AP(s) of interest. Overnight cultures of single colonies grown in 2×YT media supplemented with maintenance antibiotics were diluted 1000-fold into fresh 2×YT media with maintenance antibiotics and grown at 37° C. with shaking at 230 RPM to OD₆₀₀˜0.6-0.8 before use. SP were serially diluted 100-fold (4 dilutions total) in H₂O. 150 μL of cells was added to 10 μL of each phage dilution and to this 1 mL of liquid (55° C.) top agar (2×YT media+0.6% agar) supplemented with 2% Bluo-gal (Gold Biotechnology) was added and mixed by pipetting up and down once. This mixture was then immediately pipetted onto one quadrant of a quartered Petri dish already containing 2 mL of solidified bottom agar (2×YT media+1.5% agar, no antibiotics). After solidification of the top agar, plates were incubated at 37° C. for 16-18 h.

Phage-assisted continuous evolution (PACE): Unless otherwise noted, PACE apparatus, including host cell strains, lagoons, chemostats, and media, were all used as previously described. To reduce the likelihood of contamination with gIII-encoding recombined SP, phage stocks were purified.

Chemically competent S2060s were transformed with AP(s) and MP6 or DP6 as described above, plated on 2×YT media+1.5% agar supplemented with 25 mM glucose (to prevent induction of mutagenesis) in addition to maintenance antibiotics, and grown at 37° C. for 18-20 h. Four colonies were picked into 1 mL DRM each in a 96-well deep well plate, and this was diluted 5-fold eight times serially into DRM. The plate was sealed with a porous sealing film and grown at 37° C. with shaking at 230 RPM for 16-18 h. Dilutions with OD₆₀₀˜0.4-0.8 were then used to inoculate a chemostat containing 80 mL DRM. The chemostat was grown to OD₆₀₀˜0.8-1.0, then continuously diluted with fresh DRM at a rate of ˜1.5 chemostat volumes/h as previously described¹⁷. The chemostat was maintained at a volume of 60-80 mL.

Prior to SP infection, lagoons were continuously diluted with culture from the chemostat at 1 lagoon volume/h and pre-induced with 10 mM arabinose for at least 2 h. If DP6 was used, the lagoons were also pre-induced with aTc. Lagoons were infected with SP at a starting titer of 10⁶ pfu/mL and maintained at a volume of 15 mL. Samples (500 μL) of the SP population were taken at indicated times from lagoon waste lines. These were centrifuged at 8000 g for 2 min, and the supernatant was passed through a 0.22 μm PVDF Ultrafree centrifugal filter (Millipore) and stored at 4° C. Lagoon titers were determined by plaque assays using S2060 cells transformed with pJC175e. For Sanger sequencing of lagoons, single plaques were PCR amplified using primers AB1793 (5′- (5′-TAATGGAAACTTCCTCATGAAAAAGTCTTTAG; SEQ ID NO: 40) and AB1396 (5′-ACAGAGAGAATAACATAAAAACAGGGAAGC; SEQ ID NO: 41), both of which anneal to regions of the phage backbone flanking the evolving gene of interest. Generally, eight plaques were picked and sequenced per lagoon.

Activity Independent SE-PACE of rAPOBEC1: Host cells transformed with pTW084b, pTW051d, and DP6 were maintained in a 40 mL chemostat. The lagoon was cycled at 1 volume/h with 10 mM arabinose and 20 ng/mL aTc for 4 h prior to infection with SP30. Upon infection, lagoon dilution rates were decreased to 0.5 volume/h. At 18 h, the aTc concentration was decreased to 0 ng/mL. The lagoon dilution rate was increased to 1 volume/h at 28 h and 1.5 volumes/h at 66 h. The experiment ended at 74 h.

Evolution was continued on host cells transformed with pTW084b, pTW051d, and MP6 in an 80 mL chemostat. Lagoons were infected with a 1:1 ratio of 35.1 and 35.2, SP clones isolated from the previous experiment. Upon infection, lagoon dilution rates were maintained at 1 volume/h. The lagoon dilution rate was increased to 1.5 volume/h at 18 h, 2 volumes/h at 66 h, and 3 volumes/h at 90 h. The experiment ended at 112 h.

Evolution was continued on host cells transformed with pTW084b, pTW051b4, and MP6 in an 80 mL chemostat. Lagoons were infected with either 36.1 or 36.2, SP clones isolated from the previous experiment. Upon infection, lagoon dilution rates were decreased to 0.5 volume/h. The lagoon dilution rate was increased to 1 volume/h at 17 h, 1.5 volumes/h at 41 h, and 2 volumes/h at 66 h. At 96 h, the chemostat was replaced with a fresh 80 mL chemostat containing host cells transformed with pTW084b, pTW051d2, and MP6, and the lagoon dilution rate was decreased to 1 volume/h. The lagoon dilution rate was increased to 1.5 volumes/h at 115 h and 2 volumes/h at 168 h. The experiment ended at 184 h.

Small-scale protein expression: BL21 DE3 cells (New England BioLabs) were transformed with the expression plasmids (EPs) of interest according to manufacturer protocol. Overnight cultures of single colonies grown in 2×YT media supplemented with maintenance antibiotics were diluted 1000-fold into fresh 2×YT media (2 mL) with maintenance antibiotics and grown at 37° C. with shaking at 230 RPM to OD₆₀₀˜0.4-0.6 before induction with 1 mM isopropyl-β-D-thiogalactoside (IPTG; Gold Biotechnology) or rhamnose (Gold Biotechnology). Cells were grown for a further 3 h at 37° C. with shaking at 230 RPM. Cells from 1.4 mL of culture were isolated by centrifugation at 8000 g for 2 min. The resulting pellet was resuspended in 150 μL B-per reagent (Thermo Fisher Scientific) supplemented with protease inhibitor cocktail (Roche) and incubated on ice for 10 min before centrifugation at 16,000 g for 2 min. The supernatant was collected as the soluble fraction and the resulting pellet was resuspended in an additional 150 μL B-per reagent to obtain the insoluble fraction. To 37.5 μL of each fraction was added 12.5 μL 4×Laemmli sample loading buffer (Bio-Rad) containing 2 mM Dithiothreitol (DTT; Sigma Aldrich). After vortexing, the fractions were incubated at 95° C. for 10 min. 12 μL of each soluble fraction and 6 μL of each insoluble fraction was loaded into the wells of a Bolt 4-12% Bis-Tris Plus (Thermo Fisher Scientific) pre-cast gel. 6 μL of Precision Plus Protein Dual Color Standard (Bio-Rad) was used as a reference. Samples were separated by electrophoresis at 180 V for 35 min in Bolt MES SDS running buffer (Thermo Fisher Scientific). Gels were stained with InstantBlue reagent (Expedcon) for 1 h to overnight, then washed several times with H₂O before imaging with a G:Box Chemi XRQ (Syngene). Band densities were quantified using ImageJ and normalized to reference bands to control for protein loading.

Medium-scale protein expression and purification: BL21 DE3 cells were transformed with the EPs of interest according to manufacturer protocol. Overnight cultures of single colonies grown in 2×YT media supplemented with maintenance antibiotics were diluted 1000-fold into fresh 2×YT media (250 mL) with maintenance antibiotics and grown at 37° C. with shaking at 230 RPM to OD₆₀₀˜0.4-0.6. Cells were chilled on ice for 1 h, then induced with 1 mM IPTG and grown for a further 16-18 h at 16° C. with shaking at 200 RPM. For 37° C. post-induction growth, the cold shock step was omitted and the cells were grown for a further 3 h at 37° C. with shaking at 200 RPM after induction with 1 mM IPTG. Cells were isolated by centrifugation at 8000 g for 10 min. The resulting pellet was resuspended in 4 mL B-per reagent supplemented with EDTA-free protease inhibitor cocktail (Roche) and incubated on ice for 20 min before centrifugation at 12,000 g for 15 min. The supernatant was decanted into a 15 mL conical tube and incubated with 250 μL of TALON Cobalt (Clontech) resin at 4° C. with constant agitation for 1-2 h, after which the resin was isolated by centrifugation at 500 g for 5 min. The supernatant was decanted, and the resin resuspended in 2 mL binding buffer (50 mM NaH₂PO₄, 300 mM NaCl, 20 mM imidazole, pH 7.8) and transferred to a column. The resin was washed 4× with 1 mL binding buffer before protein was eluted with 1 mL of binding buffer containing increasing concentrations of imidazole (50-250 mM in 50 mM increments). The fractions were analyzed by SDS-PAGE for purity. Combined fractions were buffer-exchanged with TBS (20 mM Tris-Cl, 500 mM NaCl, pH 7.5) and concentrated using an Amicon Ultra-15 centrifugal filter unit (10,000 molecular weight cutoff; Millipore), then stored at 4° C. until further use. Proteins were quantified using Quick Start Bradford reagent (Bio-Rad) using BSA standards (Bio-Rad).

Medium-scale BE3 expression and purification: BE3 variants were expressed and purified. BL21 DE3 cells were transformed with the BE3-expressing EPs of interest according to manufacturer protocol. Overnight cultures of single colonies grown in 2×YT media supplemented with maintenance antibiotics were diluted 1000-fold into fresh 2×YT media (200 mL) with maintenance antibiotics and grown at 37° C. with shaking at 230 RPM to OD₆₀₀˜0.7-0.8. Cells were chilled on ice for 1 h, then induced IPTG and grown for a further 16-18 h at 16° C. with shaking at 200 RPM. Cells were isolated by centrifugation at 8000 g for 15 min. The resulting pellet was resuspended in 8 mL high salt buffer (100 mM Tris-Cl, 1 M NaCl, 5 mM tris(2-carboxyethyl)phosphine (TCEP; Gold Biotechnology), 20% glycerol, pH 8.0) supplemented with EDTA-free protease inhibitor cocktail and 1 mM phenylmethane sulfonyl fluoride (PMSF; Sigma-Aldrich). Cells were sonicated on ice (3 s on/3 s off; 6 min total) and the lysate centrifuged at 16,000 g for 15 min. The supernatant was decanted into a 15 mL conical tube and incubated with 500 μL of TALON Cobalt resin at 4° C. with constant agitation for 1 h, after which the resin was isolated by centrifugation at 500 g for 5 min. The resin was washed 5× with 1 mL high salt buffer, then eluted with 1 mL of elution buffer (100 mM Tris-Cl, 500 mM NaCl, 5 mM TCEP, 200 mM imidazole, 20% glycerol, pH 8.0). The isolated protein was then buffer-exchanged with medium salt buffer (100 mM Tris-Cl, 500 mM NaCl, 5 mM TCEP, 20% glycerol, pH 8.0) and concentrated using an Amicon Ultra-15 centrifugal filter unit (100,000 molecular weight cutoff). Proteins were quantified using Quick Start Bradford reagent using BSA standards.

Rifampicin resistance assay: BL21 DE3 cells were transformed with the EPs of interest according to manufacturer protocol. Overnight cultures of single colonies grown in DRM media supplemented with maintenance antibiotics were diluted 1000-fold into DRM media with maintenance antibiotics in a 96-well deep well plate. The plate was sealed with a porous scaling film and grown at 3720 C. with shaking at 230 RPM for until the culture reached OD₆₀₀˜0.4. The cells were then either induced with 5 mM rhamnose or repressed with 5 mM glucose before incubation for an additional 16-18 h at 37° C. with shaking at 230 RPM. Cultures were serially diluted on 2×YT+1.5% agar plates supplemented with 50 μg/mL spectinomycin, 100 μg/mL rifampin (Alfa Aesar), and 25 mM glucose. The total number of colony-forming units (cfus) was determined by serially diluting the same cultures on 2×YT+1.5% agar plates supplemented with 50 μg/mL spectinomycin and 25 mM glucose. Plates were grown at 37° C. for 16-18 h. The surviving colonies on the plates containing rifampin were counted and this number was normalized to the total cfu count.

High-throughput sequencing of genomic DNA: Genomic sites were amplified with primers targeting the region of interest and the appropriate universal Illumina forward and reverse adapters. 25 μL scale PCR 1 reactions used 1.25 μL each of 10 μM forward and reverse primers and 0.5 μL genomic DNA extract, all diluted to 12.5 μL with nuclease-free water, and 12.5 μL Phusion U Green Multiplex PCR MasterMix (Thermo Fisher Scientific). PCR 1 conditions: 98 ° C. for 2 min, then 30 cycles of (98 ° C. for 15 s, 61 ° C. for 20 s, 72 ° C. for 15 s), followed by a final 72 ° C. extension for 2 min. PCR products were verified by comparison with DNA standards (Quick-Load 2-Log Ladder; New England BioLabs) on a 2% agarose gel supplemented with ethidium bromide. Unique Illumina barcoding primers which anneal to the universal Illumina adapter region were subsequently appended to each PCR 1 sample in a second PCR reaction (PCR 2). PCR 2 reactions used 1.25 μL each of 10 μM forward and reverse Illumina barcoding primers and 1 μL of unpurified PCR 1 reaction product, all diluted to 12.5 μL with nuclease-free water, and 12.5 μL Phusion U Green Multiplex PCR MasterMix (Thermo Fisher Scientific). PCR 2 conditions: 98° C. for 2 min, then 12 cycles of (98° C. for 15 s, 61° C. for 20 s, 72° C. for 20 s), followed by a final 72° C. extension for 2 min. PCR products were pooled and purified by electrophoresis with a 2% agarose gel using a Monarch DNA Gel Extraction Kit (New England BioLabs) eluting with 30 μL H₂O. DNA concentration was quantified with the KAPA Library Quantification Kit-Illumina (KAPA Biosystems) and sequenced on an Illumina MiSeq instrument (paired-end read—R1: 220 cycles, R2: 0 cycles) according to the manufacturer's protocols.

General HTS data analysis: Sequencing reads were demultiplexed in MiSeq Reporter (Illumina). Alignment of amplicon sequences to a reference sequence was performed using a custom Matlab script. In brief, the Smith-Waterman algorithm was used to align sequences without indels to a reference sequence; bases with a quality score of less than 30 were converted to ‘N’ to prevent base miscalling as a result of sequencing error. Indels were quantified separately using a modified version of a previously described Matlab script in which sequencing reads with more than half the base calls below a quality score of Q30 were filtered out. Indels were counted as reads which contained insertions or deletions of greater than or equal to 1 bp within a 30 bp window surrounding the predicted Cas9 cleavage site.

Base editing values are representative of N=4 independent biological replicates collected over different days, with the mean±s.e.m shown. Base editing values are reported as a percentage of the number of reads with cytidine mutagenesis over the combined number of aligned reads and indel-containing reads.

Data availability: Selection plasmids used in this example will be available through Addgene and are described in Table 2.

### Evolution of rAPOBEC1

This example relates to a phage-assisted continuous evolution (PACE) system for rapidly evolving proteins with improved soluble protein expression in E. coli, either in the presence or absence of a simultaneous selection for protein function. The system uses an AND logic gate that enables PACE under two simultaneous positive selections. In particular, this example describes activity-independent evolution of APOBEC1 cytidine deaminase variants characterized by improved solubility, enhanced purification yields, and editing activity of base editors in E. coli and mammalian cells.

In some embodiments, an activity-independent selection for improving protein expression provides a useful complement to a dual (e.g., positive and negative) selection PACE system. Here, activity-independent, soluble expression PACE (SE-PACE) methods were used to address the primary source of “cheating” observed with evolution based on split T7 RNAP folding reporter—the formation of premature truncation products upstream of translation—initiating Met residues.

One embodiment of SE-PACE is depicted in FIGS. 1A-1B. Briefly, a protein of interest (POI) is fused between an affinity tag (e.g., a GCN4 peptide) and an E. coli T7 RNA polymerase variant—E. coli RNA polymerase omega subunit fusion protein (T7n-rpoZ), allowing both the protein expression and protein binding selections to operate (FIG. 1A). As the PACE selection phages (SP) carry the GCN4 peptide linked to rpoZ, they begin PACE with the ability to pass the protein binding selection. Any SP that cheat by forming truncations in the POI lose the ability to propagate on the protein binding selection, as the GCN4 peptide is no longer connected to rpoZ, and thus cheating phages are washed out of the lagoon (FIG. 1B).

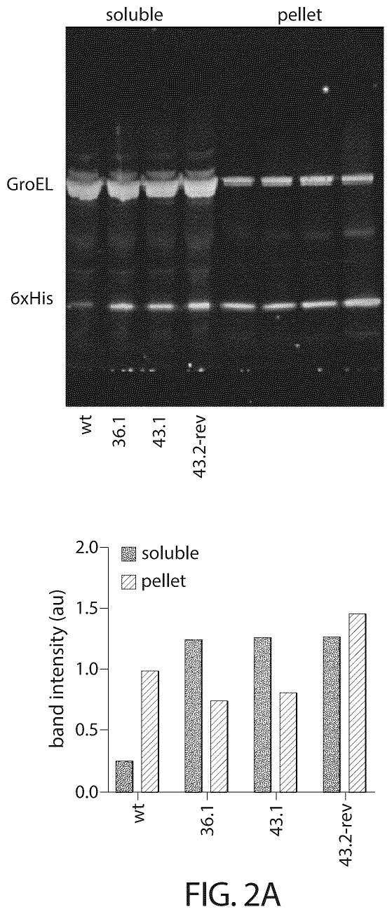

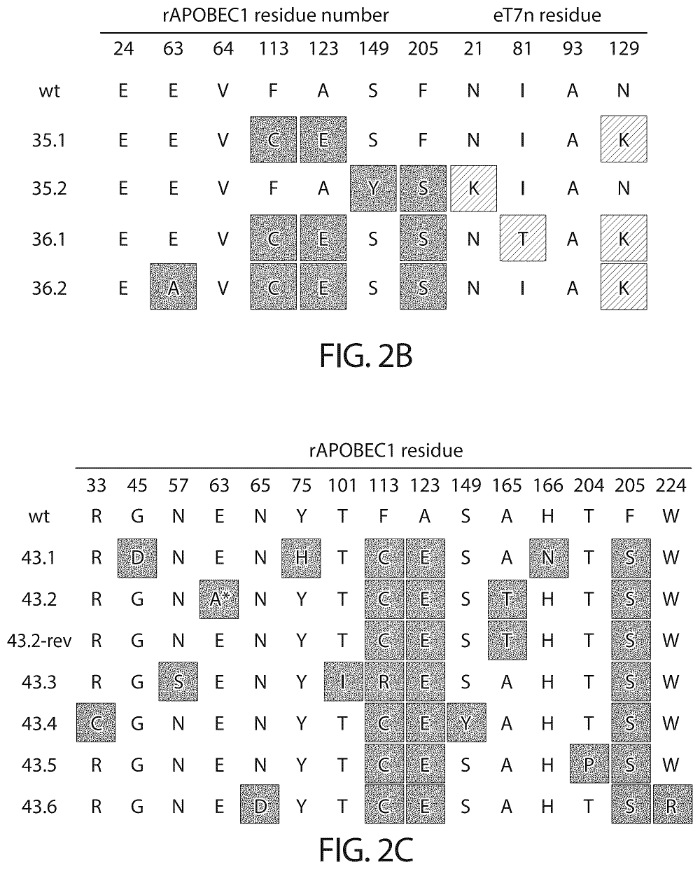

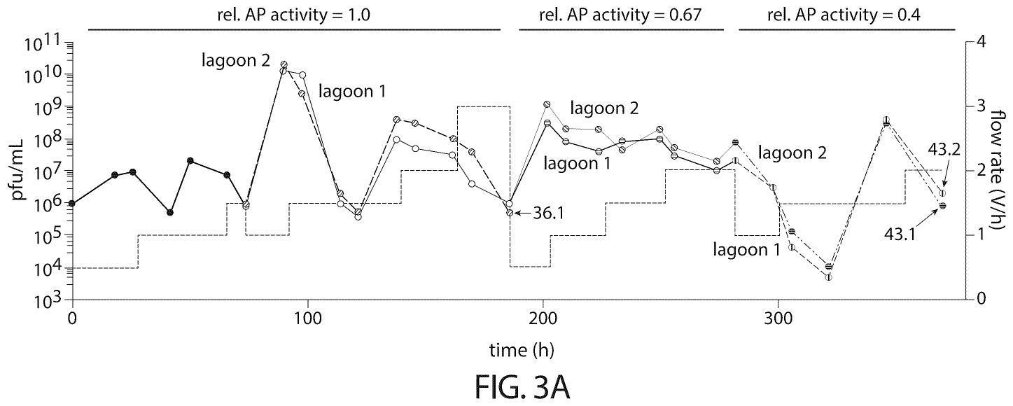

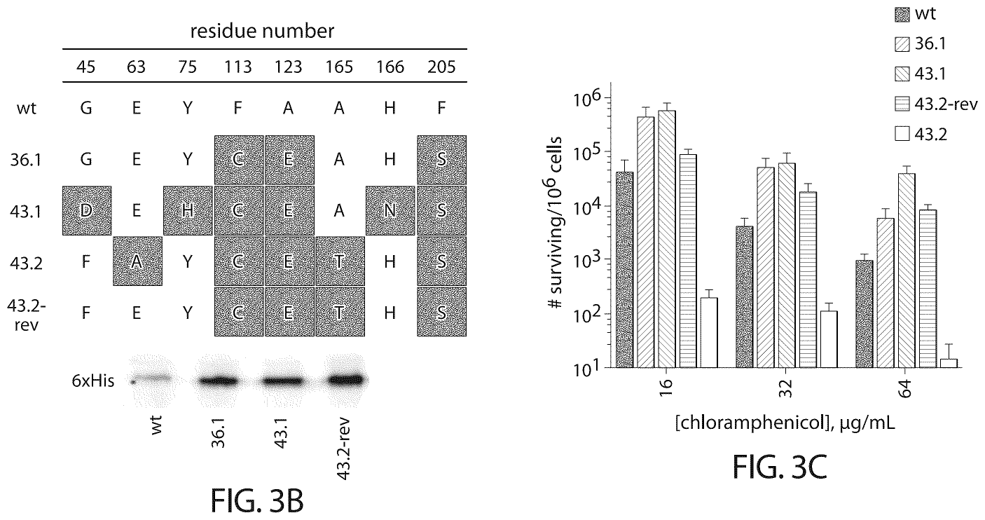

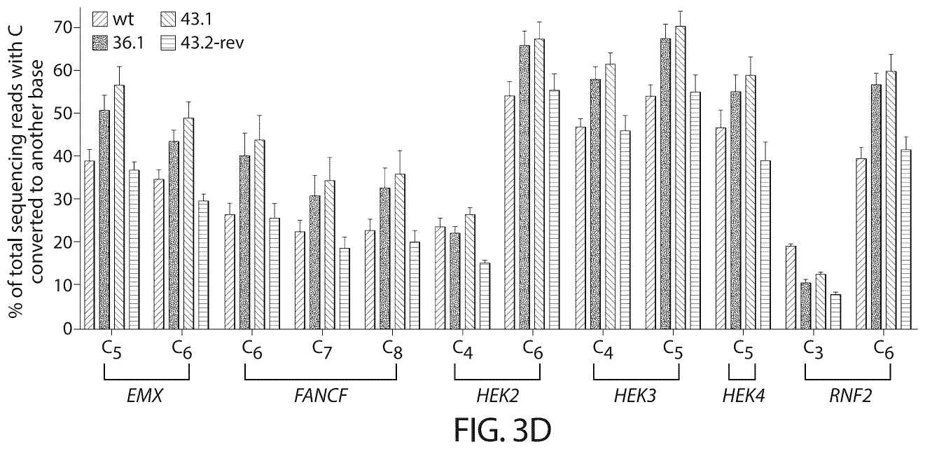

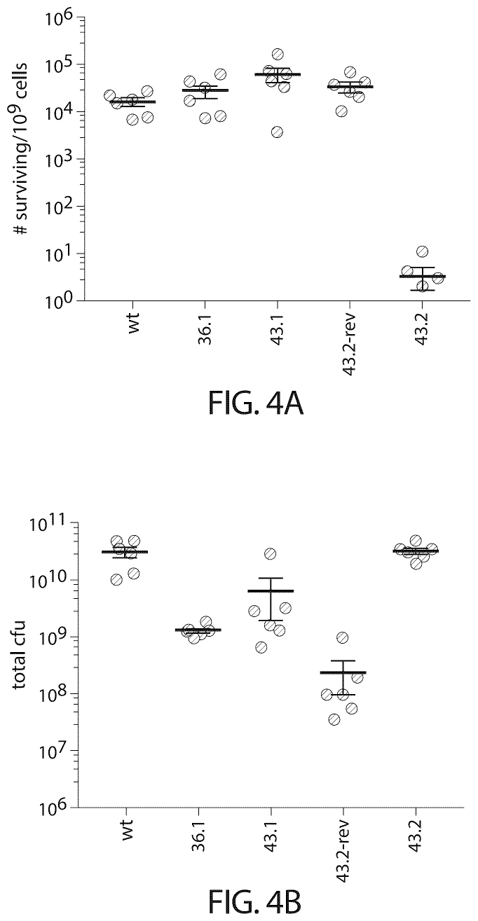

SE-PACE was used to improve the soluble expression of rat apolipoprotein B mRNA editing catalytic subunit 1 (rAPOBEC1). APOBEC1 is a potent cytidine deaminase that can act on both RNA and DNA. Like many eukaryotic proteins, rAPOBEC1 expresses poorly in E. coli and localizes almost exclusively to the insoluble fraction (FIG. 2A). SP encoding rAPOBEC1 were evolved using SE-PACE for a total of 370 h (260 average generations) using three protein expression accessory plasmids (APs) of increasing stringency (FIG. 3A). Consensus mutations from SP isolated in lagoons 1 and 2 of the first two SE-PACE experiments are shown in FIG. 2B. Mutations in rAPOBEC1 after 370 hours of SE-PACE are shown in FIG. 2C. After the first 74 h, SPs containing the two dominant genotypes were pooled and this mixture was used to infect two parallel, identically-run lagoons. At 186 h, both lagoons converged on mutations (F113C+A123E+F205S) that yielded the 36.1 genotype (FIG. 3B); however, lagoon 1 had also collected an inactivating mutation (E63A). The final 184 h of PACE produced two variants (43.1 and 43.2) that enhanced expression>4-fold (FIG. 3B and FIG. 2A). As 43.2 was identified from lagoon 1, it also carried the E63A mutation, which was reverted to obtain 43.2-rev (FIG. 3B). When tested in a rifampicin resistance assay, neither 36.1 nor 43.1 showed defects in apparent deaminase activity. Evolved APOBEC clone 43.2-rev also showed comparable activity to wild-type rAPOBEC1, while 43.2 was completely inactive (FIG. 4A). Clones 36.1, 43.1, and 43.2-rev were all observed to have a negative impact on cell growth rate when expressed, while wild-type rAPOBEC1 and 43.2 exhibited no such effect, indicating that the observed growth inhibition may be due to increased expression of active deaminase (FIG. 4B).

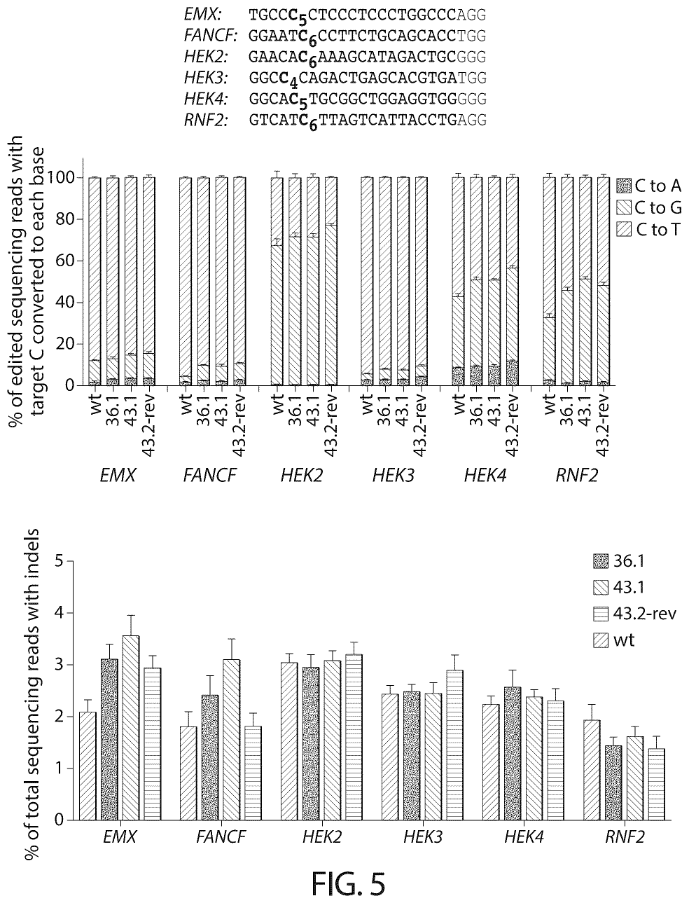

The SE-PACE-evolved APOBEC1 variants were also observed to have increased soluble expression levels in E. coli when incorporated into “base editors”, engineered genome editing proteins consisting of a fusion of rAPOBEC1, a catalytically impaired Cas9, and a uracil glycosylase inhibitor (ugi) that enable targeted conversion of individual DNA base pairs in living cells. Substitution of wild-type rAPOBEC1 with 36.1, 43.1, and 43.2-rev in the base editor BE3 improved soluble expression yields by 2-3 fold (Table 1), despite the fact that rAPOBEC1 only accounts for ˜15% of the total BE3 protein by molecular weight. Base editors incorporating evolved APOBEC variants 36.1, 43.1, and 43.2-rev also exhibited higher apparent editing activity in E. coli, as measured by the ability of BE2 (which contains dCas9 in place of Cas9 nickase) to rescue an active site mutation (H193R) in chloramphenicol acetyl transferase (FIG. 3C). Increased cytidine deamination was also observed in HEK293T cells transfected with BE3 variants 36.1 and 43.1 (FIG. 3D), albeit with modestly decreased product purities (FIG. 5). Together, these data indicate that SE-PACE improves base editor expression in E. coli.

TABLE 1
Yields of purified BE3 variants

| | Yield (mg) ^{a} | |
|---|---|---|
| protein | Replicate 1 | Replicate 2 |
| BE3 (wt) | 0.25 | 0.21 |
| 36.1-BE3 | 0.45 | 0.33 |
| 43.1-BE3 | 0.71 | 0.54 |
| 43.2-rev-BE3 | 0.56 | 0.39 |

^{a} Yield isolated from 200 mL culture in 2xYT at 16° C.

TABLE 2
Plasmids used in the Example

| | Class | | ORF1 | | ORF2 | | ORF3 | |
|---|---|---|---|---|---|---|---|---|
| Name | (res) | Origin | Prom | [RBS]¹ Genes | Prom | [RBS] Genes | Prom | [RBS] Genes |
| pTW006a | AP | SC101 | P_{T7} | [SD8] luxAB | P_{BAD} | [SD8] T7c | P_{c} | araC |
| | (carb^{R}) | | | | | | | |
| pTW006a2 | AP | SC101 | P_{T7} | [SD8] luxAB | P_{BAD} | [SD8] T7c | P_{c} | araC |
| | (carb^{R}) | | | | | R632S | | |
| pTW006a3 | AP | SC101 | P_{T7} | [SD8] luxAB | P_{BAD} | [SD8] T7c | P_{c} | araC |
| | (carb^{R}) | | | | | L637A | | |
| pTW006a4 | AP | SC101 | P_{T7} | [SD8] luxAB | P_{BAD} | [SD8] T7c | P_{c} | araC |
| | (carb^{R}) | | | | | Y639F | | |
| pTW006a5 | AP | SC101 | P_{T7} | [SD8] luxAB | P_{BAD} | [SD8] T7c | P_{c} | araC |
| | (carb^{R}) | | | | | Q649S | | |
| pTW006a6 | AP | SC101 | P_{T7} | [SD8] luxAB | P_{BAD} | [SD8] T7c | P_{c} | araC |
| | (carb^{R}) | | | | | F644A | | |
| pTW006aP1a | AP | SC101 | P_{T7} | [SD8] gIII, luxAB | Pₚᵣₒ₁ | [SD8] T7c | | |
| | (carb^{R}) | | | | | | | |
| pJC175e | AP | SC101 | Pₚₛₚ | [SD8] gIII, luxAB | | | | |
| | (carb^{R}) | | | | | | | |
| pTW026a3c | AP | SC101 | Pₚₛₚ | [SD8] gIII-N | | | | |
| | (carb^{R}) | | | | | | | |
| pTW048a3 | AP | SC101 | P_{lacZ-opt} | [sd5] gIII-N | Pₚᵣₒ₁² | 434cI − SH2_{ABL1} | | |
| | (carb^{R}) | | (OR1 + 2) | | | | | |
| pTW055a3 | AP | SC101 | P_{lacZ-opt} | [sd8] gIII-N | Pₚᵣₒ₁ | 434cI − GCN4 | | |
| | (carb^{R}) | | (OR1 + 2) | | | 7P14P | | |
| pTW074c | AP | SC101 | P_{lacZ-opt} | [sd5] gIII-N | Pₚᵣₒ₁ | 434cI − htt 1-17 | | |
| | (carb^{R}) | | (OR1) | | | | | |
| pTW084b | AP | SC101 | P_{lacZ-opt} | [sd5] gIII-N | Pₚᵣₒ₁ | 434cI − m3 | | |
| | (carb^{R}) | | (OR1 + 2) | | | | | |
| pTW032b2c | AP | ColE1 | Pₚₛₚ | [sd5] gIII-C | | | | |
| | (spec^{R}) | | | | | | | |
| pTW051a | AP | ColE1 | P_{T7} | [sd5] gIII-C | Pₚᵣₒ₁ | T7c | | |
| | (spec^{R}) | | | | | | | |
| pTW051b2 | AP | ColE1 | P_{T7a} | [sd5] gIII-C | Pₚᵣₒ₁ | T7c R632S | | |
| | (spec^{R}) | | | | | | | |
| pTW051b4 | AP | ColE1 | P_{T7d} | [sd5] gIII-C | Pₚᵣₒ₁ | T7c R632S | | |
| | (spec^{R}) | | | | | | | |
| pTW051d | AP | ColE1 | P_{T7a} | [sd5] gIII-C | Pₚᵣₒ₁ | T7c | | |
| | (spec^{R}) | | | | | R632S + Q649S | | |
| pTW051d2 | AP | ColE1 | P_{T7d} | [sd5] gIII-C | Pₚᵣₒ₁ | T7c | | |
| | (spec^{R}) | | | | | R632S + Q649S | | |
| pTW004a | CP | ColE1 | Pₜₑₜ | [SD8] MBP − T7n | | | | |
| | (spec^{R}) | | | | | | | |
| pTW004b | CP | ColE1 | Pₜₑₜ | [SD8] MBP | | | | |
| | (spec^{R}) | | | G32D + I33P − T7n | | | | |
| pTW004d | CP | ColE1 | Pₜₑₜ | [SD8] MBP V8G − | | | | |
| | (spec^{R}) | | | T7n | | | | |
| pTW004e | CP | ColE1 | Pₜₑₜ | [SD8] MBP | | | | |
| | (spec^{R}) | | | G19C − T7n | | | | |
| pTW004f | CP | ColE1 | Pₜₑₜ | [SD8] MBP | | | | |
| | (spec^{R}) | | | A276G − T7n | | | | |
| pTW004g | CP | ColE1 | Pₜₑₜ | [SD8] MBP | | | | |
| | (spec^{R}) | | | Y283D − T7n | | | | |
| pTW004h | CP | ColE1 | Pₜₑₜ | [SD8] MBP | | | | |
| | (spec^{R}) | | | T345I − T7n | | | | |
| pTW035d | EP | pBR322 | P_{T7lac} | 6xHis − MBP | | | | |
| | (kan^{R}) | | | G32D + I33P | | | | |
| pTW035e | EP | pBR322 | P_{T7lac} | 6xHis − MBP | | | | |
| | (kan^{R}) | | | | | | | |
| pTW035g | EP | pBR322 | P_{T7lac} | 6xHis − MBP V8G | | | | |
| | (kan^{R}) | | | | | | | |
| pTW035h | EP | pBR322 | P_{T7lac} | 6xHis − MBP | | | | |
| | (kan^{R}) | | | G19C | | | | |
| pTW035i | EP | pBR322 | P_{T7lac} | 6xHis − MBP | | | | |
| | (kan^{R}) | | | A276G | | | | |
| pTW035j | EP | pBR322 | P_{T7lac} | 6xHis − MBP | | | | |
| | (kan^{R}) | | | Y283D | | | | |
| pTW035b | EP | pBR322 | P_{T7lac} | 6xHis − MBP | | | | |
| | (kan^{R}) | | | P33T + T275I | | | | |
| pTW035c | EP | pBR322 | P_{T7lac} | 6xHis − MBP | | | | |
| | (kan^{R}) | | | P33T + V76I + | | | | |
| | | | | A167V + V373I | | | | |
| pTW035aa | EP | pBR322 | P_{T7lac} | 6xHis − MBP | | | | |
| | (kan^{R}) | | | D33T + F258Y | | | | |
| pTW035ab | EP | pBR322 | P_{T7lac} | 6xHis − MBP D33L | | | | |
| | (kan^{R}) | | | | | | | |
| pTW035ac | EP | pBR322 | P_{T7lac} | 6xHis − MBP | | | | |
| | (kan^{R}) | | | G24V + D33S | | | | |
| pTW069a | EP | pBR322 | P_{T7lac} | 6xHis − Ωg | | | | |
| | (kan^{R}) | | | | | | | |
| pTW069h | EP | pBR322 | P_{T7lac} | 6xHis − m3 | | | | |
| | (kan^{R}) | | | | | | | |
| pTW075I | EP | pBR322 | P_{T7lac} | 6xHis − 29.1.2 | | | | |
| | (kan^{R}) | | | | | | | |
| pTW075k | EP | pBR322 | P_{T7lac} | 6xHis − 29.1.5 | | | | |
| | (kan^{R}) | | | | | | | |
| pTW087a | EP | pBR322 | P_{T7lac} | 6xHis − MBP − | | | | |
| | (kan^{R}) | | | TEV − GCN4 | | | | |
| | | | | 7P14P | | | | |
| pTW081a | EP | pBR322 | P_{T7lac} | 6xHis − C4 | | | | |
| | (kan^{R}) | | | | | | | |
| pTW081d | EP | pBR322 | P_{T7lac} | 6xHis − C4 V38F | | | | |
| | (kan^{R}) | | | | | | | |
| pTW081e | EP | pBR322 | P_{T7lac} | 6xHis − C4 A98V | | | | |
| | (kan^{R}) | | | | | | | |
| pTW081i | EP | pBR322 | P_{T7lac} | 6xHis − 34.1.2 | | | | |
| | (kan^{R}) | | | | | | | |
| pTW081j | EP | pBR322 | P_{T7lac} | 6xHis − 34.2.3 | | | | |
| | (kan^{R}) | | | | | | | |
| pTW081k | EP | pBR322 | P_{T7lac} | 6xHis − 34.2.6 | | | | |
| | (kan^{R}) | | | | | | | |
| pTW101a | EP | ColE1 | rhaP_{BAD} | 6xHis − | rhaP_{RS} | rhaS, rhaR | | |
| | (spec^{R}) | | | rAPOBEC1 | | | | |
| pTW101h | EP | ColE1 | rhaP_{BAD} | 6xHis − 36.1 | rhaP_{RS} | rhaS, rhaR | | |
| | (spec^{R}) | | | | | | | |
| pTW101t | EP | ColE1 | rhaP_{BAD} | 6xHis − 43.1 | rhaP_{RS} | rhaS, rhaR | | |
| | (spec^{R}) | | | | | | | |
| pTW101m | EP | ColE1 | rhaP_{BAD} | 6xHis − 43.2 | rhaP_{RS} | rhaS, rhaR | | |
| | (spec^{R}) | | | | | | | |
| pTW101m2 | EP | ColE1 | rhaP_{BAD} | 6xHis − 43.2-rev | rhaP_{RS} | rhaS, rhaR | | |
| | (spec^{R}) | | | | | | | |
| pHR41¹³ | EP | pBR322 | P_{T7lac} | 6xHis − BE3 | | | | |
| | (kan^{R}) | | | | | | | |
| pTW142a | EP | pBR322 | P_{T7lac} | 6xHis − 36.1-BE3 | | | | |
| | (kan^{R}) | | | | | | | |
| pTW142b | EP | pBR322 | P_{T7lac} | 6xHis − 43.1-BE3 | | | | |
| | (kan^{R}) | | | | | | | |
| pTW142c | EP | pBR322 | P_{T7lac} | 6xHis − 43.2-rev- | | | | |
| | (kan^{R}) | | | BE3 | | | | |
| pNMG98¹⁴ | EP | SC101 | P_{lac} | gRNA | P_{BAD} | BE2 | P_{c} | araC |
| | (carb^{R}) | | | | | | | |
| pTW143a | EP | SC101 | P_{lac} | gRNA | P_{BAD} | 36.1-BE2 | P_{c} | araC |
| | (carb^{R}) | | | | | | | |
| pTW143b | EP | SC101 | P_{lac} | gRNA | P_{BAD} | 43.1-BE2 | P_{c} | araC |
| | (carb^{R}) | | | | | | | |
| pTW143c | EP | SC101 | P_{lac} | gRNA | P_{BAD} | 43.2-rev-BE2 | P_{c} | araC |
| | (carb^{R}) | | | | | | | |
| pTW143d | EP | SC101 | P_{lac} | gRNA | P_{BAD} | 43.2-BE2 | P_{c} | araC |
| | (carb^{R}) | | | | | | | |
| antibiotic | spec^{R} | RSF1030 | Pₖₐₙ | kan^{R} | P_{cat} | cam^{R} H193R | | |
| selection | | | | | | | | |
| uracil¹⁴ | | | | | | | | |
| PACK129¹⁵ | carb^{R} | pBR322 | P_{CMV} | BE3 | | | | |
| pTW107a | carb^{R} | pBR322 | P_{CMV} | 36.1 BE3 | | | | |
| pTW107b | carb^{R} | pBR322 | P_{CMV} | 43.1 BE3 | | | | |
| pTW107c | carb^{R} | pBR322 | P_{CMV} | 43.2-rev BE3 | | | | |
| MP6 | MP | cloDF13 | P_{BAD} | dnaQ926, dam, | P_{c} | araC | | |
| | (chlor^{R}) | | | seqA, emrR, ugi, | | | | |
| | | | | cda1 | | | | |
| DP6 | MP | cloDF13 | P_{BAD} | dnaQ926, dam, | P_{c} | araC | Pₚₛₚ₋ₜₑₜ | [sd8] gIII |
| | (chlor^{R}) | | | seqA, emrR, ugi, | | | | |
| | | | | cda1 | | | | |
| SP01a | SP | M13 f1 | P_{gIII} | MBP − T7n | | | | |
| | (none) | | | | | | | |
| SP01z | SP | M13 f1 | P_{gIII} | T7n | | | | |
| | (none) | | | | | | | |
| SP02a | SP | M13 f1 | P_{gIII} | MBP G32D + | | | | |
| | (none) | | | I33P − T7n | | | | |
| SP02b | SP | M13 f1 | P_{gIII} | MBP Y283D − T7n | | | | |
| | (none) | | | | | | | |
| SP09b3 | SP | M13 f1 | P_{gIII} | HA4 − T7n − rpoZ | | | | |
| | (none) | | | | | | | |
| SP10b2 | SP | M13 f1 | P_{gIII} | HA4 Y87A − T7n − | | | | |
| | (none) | | | rpoZ | | | | |
| SP16c3 | SP | M13 f1 | P_{gIII} | Ωg − eT7n − rpoZ | | | | |
| | (none) | | | | | | | |
| SP24a3 | SP | M13 f1 | P_{gIII} | C4 − eT7n − rpoZ | | | | |
| | (none) | | | | | | | |
| SP27b | SP | M13 f1 | P_{gIII} | GCN4 7P14P − | | | | |
| | (none) | | | MBP G32D + | | | | |
| | | | | I33P − eT7n − rpoZ | | | | |
| SP30 | SP | M13 f1 | P_{gIII} | GCN4 7P14P − | | | | |
| | (none) | | | rAPOBEC1 − | | | | |
| | | | | eT7n − rpoZ | | | | |
| SP39b | SP | M13 f1 | P_{gIII} | m3-noCys − | | | | |
| | (none) | | | eT7n − rpoZ | | | | |
| SP39d | SP | M13 f1 | P_{gIII} | 29.1.5-noCys − | | | | |
| | (none) | | | eT7n − rpoZ | | | | |
| SP13 | SP | M13 f1 | P_{gIII} | kan | | | | |
| | (none) | | | | | | | |
| SP98 | SP | M13 f1 | P_{gIII} | rpoZ − HA4 | | | | |
| | (none) | | | | | | | |

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

What is claimed is:

1. A cytidine deaminase protein variant comprising an amino acid sequence that is at least 90% identical to a cytidine deaminase protein, wherein the amino acid sequence includes at least one mutation at a position selected from the group consisting of R33, G45, N57, N65, Y75, T101, F113, F113, A123, S149, A164, H165, H166, T204, F205, and W224 of SEQ ID NO: 15 [rAPOBEC1].

2. The cytidine deaminase protein variant of claim 1, wherein the amino acid sequence is at least 95%, at least 96%, at least 97%, at least 98%, or at least 99% identical to SEQ ID NO: 15.

3. The cytidine deaminase protein variant of claim 1 or 2, wherein the amino acid sequence includes one or more of the following mutations: R33C, G45D, N57S, N65D, Y75H, T101I, F113C, F113R, A123E, S149Y, A164T, H165N, H166N, T204P, F205S, or W224R.

4. The cytidine deaminase protein variant of any one of claims 1 to 3, wherein the amino acid sequence of the protein does not comprise a mutation of position E63.

5. The cytidine deaminase protein variant of any one of claims 1 to 4, wherein the amino acid sequence includes mutations at positions F113, A123, and F205.

6. The cytidine deaminase protein variant of any one of claims 1 to 5, wherein the amino acid sequence includes mutations at positions F113C, A123E, and F205S.

7. The cytidine deaminase protein variant of any one of claims 1 to 6, wherein the amino acid sequence is represented by SEQ ID NO: 16.

8. The cytidine deaminase protein variant of any one of claims 1 to 4, wherein the amino acid sequence includes mutations at positions G45, Y75, F113, A123, H166, and F205.

9. The cytidine deaminase protein variant of claim 8, wherein the amino acid sequence includes mutations at positions G45D, Y75H, F113C, A123E, H166N, and F205S.

10. The cytidine deaminase protein variant of claim 8 or 9, wherein the amino acid sequence is represented by SEQ ID NO: 17.

11. The cytidine deaminase protein variant of any one of claims 1 to 4, wherein the amino acid sequence includes mutations at positions F113, A123, A165, and F205.

12. The cytidine deaminase protein variant of claim 11, wherein the amino acid sequence includes mutations at positions F113C, A123E, A165T, and F205S.

13. The cytidine deaminase protein variant of claim 11 or 12, wherein the amino acid sequence is represented by SEQ ID NO: 19.

14. An isolated nucleic acid encoding the cytidine deaminase protein variant of any one of claims 1 to 13.

15. The isolated nucleic acid of claim 14, comprising the sequence set forth in any one of SEQ ID NOs: 7, 8, or 10.

16. The isolated nucleic acid of claim 14, wherein the isolated nucleic acid is codon optimized for expression in mammalian cells.

17. The isolated nucleic acid of claim 16, comprising the sequence set forth in any one of SEQ ID NOs: 12-14.

18. A host cell comprising the cytidine deaminase protein variant of any one of claims 1 to 13 or the isolated nucleic acid of any one of claims 14 to 17.

19. The host cell of claim 18, wherein the host cell is a bacterial cell.

20. The host cell of claim 18, wherein the bacterial cell is an E. coli cell.

21. The host cell of claim 18, wherein the host cell is a mammalian cell or a human cell.

22. A fusion protein comprising: (i) a RNA-programmable nuclease; (ii) a cytosine deaminase protein variant of any one of claims 1 to 13; and (iii) a uracil glycosylase inhibitor (UGI) domain.

23. The fusion protein of claim 22, wherein the RNA-programmable nuclease is a Cas9 domain comprising an amino acid sequence that is at least 85% identical to the amino acid sequence provided in SEQ ID NO: 20 or 21.

24. The fusion protein of claim 22, wherein the Cas9 domain is a Cas9 nickase domain that cuts a nucleotide target strand of a nucleotide duplex, wherein the nucleotide target strand is the strand that binds to a gRNA of the Cas9 nickase domain.

25. The fusion protein of any one of claims 22 to 24, wherein the UGI domain comprises a domain capable of inhibiting Uracil DNA Glycosylase (UDG) activity.

26. The fusion protein of any one of claims 22 to 25, wherein the UGI domain comprises an amino acid sequence that is at least 85% identical to SEQ ID NO: 22.

27. An isolated nucleic acid comprising a nucleic acid sequence encoding the fusion protein of any one of claims 22 to 26.

28. A host cell comprising the fusion protein of any one of claims 22 to 26 or the isolated nucleic acid of claim 27.

29. A complex comprising the fusion protein of any one of claims 22 to 26, and a guide RNA (gRNA) bound to the RNA-programmable nuclease of the fusion protein.

30. The complex of claim 29, wherein the gRNA is from 15-100 nucleotides long and comprises a sequence of at least 10 contiguous nucleotides that is complementary to a target sequence.

31. A method comprising contacting a cell with the complex of claim 29 or 30, wherein the gRNA of the complex comprises a sequence of at least 10 contiguous nucleotides that is complementary to a target sequence in the genome of an organism and comprises a target base pair.

32. The method of claim 31, wherein the target base pair comprises a T to C point mutation associated with a disease or disorder, and wherein the deamination of the mutant C base results in a sequence that is not associated with a disease or disorder.

33. The method of claim 31 or 32, wherein the cell is a human cell.

34. The method of any one of claims 31 to 33, wherein the cell is in vivo.

35. The method of any one of claims 31 to 33, wherein the cell is in vitro.

36. A vector system comprising:
(i) a selection phagemid comprising an isolated nucleic acid comprising an expression construct encoding a fusion protein comprising, in the following order: a GCN4 peptide, a cytidine deaminase protein, a T7 RNA polymerase, and an E. coli RNA polymerase omega subunit (rpoZ);
(ii) a first accessory plasmid comprising an isolated nucleic acid comprising an expression construct encoding a RNA polymerase I promoter operably linked to an expression cassette comprising, in the following order: a sequence encoding a M13 phage gIII protein signal peptide, and a sequence encoding a Npu split intein N-terminal portion, wherein the sequence encoding the gIII protein signal peptide lacks one or more nucleic acid bases of the signal peptide domain; and
(iii) a second accessory plasmid comprising an isolated nucleic acid comprising an expression construct encoding a T7 promoter operably linked to an expression cassette comprising, in the following order: a sequence encoding a Npu split intein C-terminal portion, and a sequence encoding a M13 phage gIII protein, wherein the sequence encoding the gIII protein lacks one or more nucleic acid bases in the signal peptide domain.

37. The vector system of claim 36, wherein the first accessory plasmid further comprises an isolated nucleic acid encoding an anti-GCN4 binding protein, optionally wherein the anti-GCN4 binding protein is an anti-GCN4 single chain variable fragment.

38. The vector system of claim 36 or 37, wherein the sequence encoding the gIII protein lacks nucleic acid bases 1-10 of the signal peptide domain.

39. The vector system of any one of claims 36 to 38, wherein the isolated nucleic acid of (iii) comprises a sequence encoding a protein as set forth in SEQ ID NO: 42.

40. The vector system of any one of claims 36 to 39, wherein the isolated nucleic acid of (ii) further comprises a sequence encoding a cI repressor protein binding site, optionally wherein the repressor protein is 434cI.

41. The vector system of any one of claims 36 to 40, wherein the cytidine deaminase protein is an APOBEC1, optionally wherein the APOBEC1 is rat APOBEC1 or human APOBEC1.

42. The vector system of any one of claims 36 to 41 further comprising a mutagenesis plasmid.

43. A kit comprising:
(i) a first container housing the selection phagemid of claim 36;
(ii) a second container housing the first accessory plasmid of claim 36; and
(iii) a third container housing the second accessory plasmid of claim 36.

44. The kit of claim 43, further comprising a container housing one or more bacterial cells, optionally wherein the bacterial cells are E. coli cells.
